(19) United States
(12) Patent Application Publication
(10) Pub. No.: US 2026/0348930 A1
(43) Pub. Date: Nov. 5, 2026

(54) ASSAY FOR DETECTION AND QUANTIFICATION OF PFAS COMPOUNDS

(71) Applicant: FREDsense Technologies Corp., Calgary (CA)

(72) Inventors: Robert Mayall, Calgary (CA); Maria Stietz, Calgary (CA); Margaret Renaud-Young, Calgary (CA); Julia Guegueniat, Calgary (CA); April Woods, Calgary (CA); Cheng Chen, Calgary (CA)

(73) Assignee: FREDsense Technologies Corp., Calgary (CA)

(21) Appl. No.: 19/699,734

(22) Filed: Jun. 5, 2026

Related U.S. Application Data
(63) Continuation of application No. PCT/CA2024/051648, filed on Dec. 12, 2024.
(60) Provisional application No. 63/610,171, filed on Dec. 14, 2023.
(60) Provisional application No. 63/679,782, filed on Aug. 6, 2024.
(60) Provisional application No. 63/689,017, filed on Aug. 30, 2024.

(51) Int. Cl. G01N 31/22; G01N 1/40; G01N 21/64; G01N 21/78; G01N 33/18

(52) U.S. Cl. CPC G01N 31/22; G01N 1/405; G01N 21/6428; G01N 21/78; G01N 33/18

(57) ABSTRACT

Methods and kits are provided for detecting and quantifying a PFAS compound or a mixture of PFAS compounds in a sample. The method includes the steps of providing a complex of a cyclodextrin and an indicator molecule, contacting the complex with the sample and measuring a parameter of the indicator molecule. The parameter is correlated with an extent of displacement of the reporter molecule from the cyclodextrin and binding of the PFAS compound to the cyclodextrin. This indicates the presence and quantity of the PFAS compound in the sample. The method is useful for monitoring samples such as environmental water samples. The kits include assay solutions for performing the methods and may also include solid-phase extraction matrices for pre-treatment of samples.

### CROSS REFERENCE TO RELATED APPLICATIONS

This application, filed under 35 U.S.C. § 111(a), is a continuation of PCT Application No. PCT/CA2024/051648, filed on Dec. 12, 2024, which claims the benefit of priority to U.S. Provisional Patent Application No. 63/610,171, filed on Dec. 14, 2023, U.S. Provisional Patent Application No. 63/679,782, filed on Aug. 6, 2024 and U.S. Provisional Patent Application No. 63/689,017 filed on Aug. 30, 2024. Each of the above-referenced patent applications is incorporated herein by reference in its entirety.

### TECHNICAL FIELD

The invention relates to detection and quantification of perfluorinated and polyfluorinated substances which are commonly known as PFAS compounds. Assays and kits for this purpose are described

### BACKGROUND

Polyfluoroalkyl and perfluoroalkyl substances (PFAS) make up a large group of persistent anthropogenic chemicals used in industrial processes and commercial products over the past 60 years. Widespread use and extreme resistance to degradation have resulted in the ubiquitous presence of these compounds in the environment. The 2011-2012 U.S. National Health and Nutrition Examination Survey reported detectable serum PFAS concentrations in virtually all individuals (97%). Human PFAS exposure has been linked to cancer, elevated cholesterol, obesity, immune suppression, and endocrine disruption. Health concerns have prompted manufacturers in Europe and North America to phase out production of some long-chain PFAS. Limited available data suggest that widespread exposure to replacement (short chain) PFASs may also adversely affect human health. Human PFAS exposure includes dietary sources, household dust, air, and drinking water. Exposure from drinking water is a serious concern because of the high aqueous solubility of many PFAS compounds. Relatively low PFAS concentrations can lead to elevated exposures in the general population. Elevated PFAS concentrations in U.S. drinking water have been reported in numerous regions, especially near industrial sites that produce or use them (Hu et al., Environ. Sci. Technol. Lett. 2016, 3, 10, 344-350, incorporated herein by reference in its entirety).

Current regulatory methods and technologies for detecting PFAS compounds can reliably quantify about 50 specific PFAS, but these technologies are unable to detect or quantify the thousands of other PFAS known to exist. These technologies typically use lab-scale analytical chemistry techniques to separate, identify and quantify PFAS substances in different water matrices (landfill leachate, industrial waste, groundwater, drinking water, and surface water). Less common approaches do not separate components prior to detection. Due to the abundant nature of PFAS and its derivatives with their uniquely specific chemical structures and properties, different methods have been used based on the species of PFAS tested. All of the different EPA methods use mass spectrometry and differ by the way the molecules are separated: liquid chromatography (LC-MS/MS), gas chromatography (GC-MS), or High-Performance liquid chromatography (HPLC) coupled with mass spectrometry. The most notable method, used in EPA 1633 and EPA 537, is LC-MS/MS (liquid chromatography with tandem mass spectrometry). By applying standards, the PFAS molecules can later be quantified. Typical detection limits for the LC-MS/MS method, combined with sample preparation using SPE cartridges, range from parts-per-billion (ppb) to low parts-per-trillion (ppt), depending on the specific PFAS compound and instrument sensitivity.

In the last 5-10 years, different processes have been developed to bring PFAS testing into the field where it is most desirable to obtain rapid results in detection and quantification of PFAS compounds. The most notable example is Starfire's Centurion® system (Starfire Industries, LLC, Champaign, IL, USA), which despite its heavy weight (350 kg), can facilitate detection at parts-per-trillion levels. The advantage of this system resides in its speed, as it would provide results within minutes. Recently, MITRE (Bedford, MA, USA), and other academic researchers have investigated development of PFAS sensors based on molecularly imprinted polymers (MIPs) (Lu et al. Sensors and Actuators B, 2022, 352, 131055; Tasfaout et al., Talanta, 2023, 258, 124434).

Some of these MIPs-based sensors have shown a very low limit of detection for specific molecules such as GenX (Glasscot et al., Crit. Rev. Anal. Chem., 2022, 52, 1258, having a limit of detection of 0.083 ppt), but no application of these sensors in the field has been disclosed to date. Metal-organic frameworks (MOFs) are used to bind PFAS molecules such as PFOS which are proposed to be used to create MOF-based sensors, using microfluidics and electrochemical detection (Cheng et al., ACS Appl. Mater. Interfaces, 2020, 12, 10503). An overview of the different methods used to detect PFAS compounds is described in Menger et al., Chem Eng. J. 2021, 417, 128133.

There is a need for improved technologies for detection of PFAS compounds, particularly detector systems which can be operated outside of laboratories.

### SUMMARY

According to one embodiment, there is provided a method for detecting and quantifying a PFAS compound or a mixture of PFAS compounds in a sample, the method comprising: providing a complex of a cyclodextrin and an indicator molecule; contacting the complex with the sample and measuring a parameter of the indicator molecule, wherein the parameter is correlated with an extent of displacement of the indicator molecule from the cyclodextrin upon binding of the PFAS compound to the cyclodextrin, thereby indicating presence and quantity of the PFAS compound or the mixture of PFAS compounds in the sample.

The PFAS compound may comprise a PFAS compound comprising at least two carbon atoms and/or the mixture of PFAS compounds may comprise a mixture of PFAS compounds, each having at least two carbon atoms.

In some embodiments, the PFAS compound or the PFAS compound of the mixture of PFAS compounds is selected from any one of or a combination of perfluorononanoic acid (PFNA), perfluorooctanoic acid (PFOA), perfluorooctane sulfonate (PFOS), perfluorohexane sulfonate (PFHxS), perfluorohexanoic acid (PFHxA), perfluorobutane sulfonate (PFBS), perfluorobutanoic acid (PFBA), perfluorotetradecanoic acid (PFTDeA), perfluoropropanesulfonic acid (PFPrS), 6:2-fluorotelomersulfonic acid (6:2 FTS); 8:2-fluorotelomersulfonic acid (8:2 FTS); perfluoro-3,6-dioxaheptanoic acid (NFDHA); perfluorodecanoic acid (PFDA); perfluoroheptanoic acid (PFHpA); perfluoroheptanesulfonic acid (PFHpS); perfluoro-4-methyoxybutanoic acid (PFMBA); perfluoro-3-methoxypropanoic acid (PFMPA); perfluoropentanoic acid (PFPeA); trifluoroacetic acid (TFA); perfluoroundecanoic acid (PFUnA) and hexafluoropropylene oxide dimer acid (HFPO-DA), or wherein the PFAS compound is a PFAS compound of an aqueous film-forming foam (AFFF).

In some embodiments, the cyclodextrin is β-cyclodextrin. The cyclodextrin may be a β-cyclodextrin polymer. The β-cyclodextrin polymer may include a linker. In some embodiments, the linker is a cyclic ether. In some embodiments, the cyclic ether is a substituted or unsubstituted oxacyclopropane, oxacyclobutane, oxacyclopentane, oxacyclohexane or furan.

In some embodiments, the β-cyclodextrin polymer comprises at least one non-hydrogen substituent on at least one glucopyranoside unit. In some embodiments, the non-hydrogen substituent is an electron withdrawing group and/or a positively charged substituent. In some embodiments, the non-hydrogen substituent is selected from any one of or a combination of: protonated primary amine, protonated secondary amine, protonated tertiary amine, quaternary amine, cyano, azido, nitro, hydroxypropyl, hydroxyamino, aldehyde, carboxyl, acetyl, ester, halogen, haloalkyl, hydroxyl, alkoxyl, and sulfonyl.

The β-cyclodextrin polymer may comprise an average of at least about 1.5 quaternary amine substituents per cyclodextrin unit, at least about 3 quaternary amine substituents per cyclodextrin unit, or at least about 4.5 quaternary amine substituents per cyclodextrin unit.

In some embodiments, the indicator molecule is a dye molecule. The dye molecule may be a colorimetric dye molecule or a fluorescent dye molecule. The fluorescent dye molecule may be 9-(diethylamino)-5H-benzo[a]phenoxazin-5-one) (Nile red), SYPRO™ Orange, 8-anilinonaphthalene-1-sulfonic acid (ANS) or bis-8-anilinonaphthalene-1-sulfonic acid (bis-ANS). In some embodiments, the fluorescent dye is provided at a concentration between about 0.0001 mM and about 0.015 mM.

In some embodiments, the sample is processed by column chromatography, and resuspension in a reduced volume of solvent to concentrate the sample prior to the step of contacting the complex with the sample.

In some embodiments, the method further comprises providing a scavenger molecule for sequestering one or more interferents present in the sample. The interferents may be compounds having at a hydrocarbon chain of a length of at least 6 carbons. The interferents may be hydrocarbons or surfactants.

In some embodiments, the scavenger molecule is α-cyclodextrin.

According to another embodiment, there is provided a use of the method described herein for determining a quantity of total PFAS compounds in a sample containing more than one PFAS compound.

According to another embodiment, there is provided a kit for preparing an assay mixture for detecting and quantifying one or more PFAS compounds in a sample. The kit comprises a mixture of assay components with the mixture comprising: a cyclodextrin; and an indicator molecule which binds to the cyclodextrin and which is displaced from the cyclodextrin by a PFAS compound. The mixture may be contained in a vessel in an aqueous solution or in dried form.

In some embodiments of the kit, the cyclodextrin is β-cyclodextrin. The cyclodextrin may be β-cyclodextrin polymer. The β-cyclodextrin polymer may comprise at least one non-hydrogen substituent on at least one glucopyranoside unit. The non-hydrogen substituent may be an electron withdrawing group and/or a positively charged substituent. The non-hydrogen substituent may be selected from any one of or a combination of: protonated primary amine, protonated secondary amine, protonated tertiary amine, quaternary amine, cyano, azido, nitro, hydroxypropyl, hydroxyamino, aldehyde, carboxyl, acetyl, ester, halogen, haloalkyl, hydroxyl, alkoxyl, and sulfonyl.

In some embodiments of the kit, the β-cyclodextrin polymer comprises an average of at least about 1.5 quaternary amine substituents per cyclodextrin unit, at least about 3 quaternary amine substituents per cyclodextrin unit, or at least about 4.5 quaternary amine substituents per cyclodextrin unit.

In some embodiments of the kit, the indicator molecule is a dye molecule. The dye molecule may be a colorimetric dye molecule or a fluorescent dye molecule. The fluorescent dye molecule is 9-(diethylamino)-5H-benzo[a]phenoxazin-5-one) (Nile red), SYPRO™ Orange, 8-anilinonaphthalene-1-sulfonic acid (ANS) or bis-8-anilinonaphthalene-1-sulfonic acid (bis-ANS).

In some embodiments, the kit further comprises a scavenger molecule for sequestering one or more interferents present in the sample. The scavenger molecule may be α-cyclodextrin.

In some embodiments of the kit, the vessel is a well of a multi-well plate, tube or cuvette, and wherein the mixture of assay components is provided in a plurality of vessels with different concentrations of the PFAS molecule provided in selected wells of the plurality of wells designated for generation of a standard curve of PFAS concentrations.

In some embodiments, the kit further comprises a solid phase extraction matrix for pre-treatment of the sample to remove interferents and/or to concentrate the one or more PFAS compounds.

In some embodiments of the kit, the solid phase extraction matrix comprises any one of or any combination of a weak anion exchange resin, a hydrophobic resin and a carbon matrix.

In some embodiments of the kit, the solid phase extraction matrix is provided in a column or in a well of a multi-well plate, or in a tube or a cuvette.

In some embodiments, the kit further comprises a wash solution for washing the solid phase extraction matrix, the wash solution comprising from about 1% to about 10% methanol.

### BRIEF DESCRIPTION OF THE DRAWINGS

In the examples section below, one or more embodiments of the present technology are described in relation to the attached figures. These embodiments are intended to provide a better understanding of the invention, how the invention may be put into practice, and to demonstrate certain advantages of the invention.

FIG. 1A is a plot of fluorescence intensity vs. concentration of β-cyclodextrin (m-β-CD) and polymeric β-cyclodextrin (p-β-CD) indicating binding of Nile Red and 8-anilino-1-naphthalenesulphonic acid (ANS) to the cyclodextrins.

FIG. 1B is a plot of fluorescence intensity vs. concentration of β-cyclodextrin indicating binding of ANS to polymeric β-cyclodextrin (p-β-CD), 2-hydroxypropyl-β-cyclodextrin (2HP-β-CD), and triacetyl-β-cyclodextrin (TAc-β-CD) using a dilution series of the β-cyclodextrins and 0.008 mM ANS.

FIG. 1C is a plot of fluorescence intensity vs. concentration of β-cyclodextrin indicating binding of ANS to a series of cyclodextrins provided at a concentration of 0.25 mM, including polymeric-β-cyclodextrin (p-β-CD), 2-hydroxypropyl-β-cyclodextrin (2HP-β-CD), triacetyl-β-cyclodextrin (TAc-β-CD), and monomeric-β-cyclodextrin (m-β-CD) using a dilution series of ANS, and compared to a control series of ANS with water.

FIG. 2 is a plot of fluorescence intensity vs. concentration of perfluorooctanoic acid (PFOA) (ppm) in a preliminary experiment of a test indicator displacement assay with displacement of ANS from p-β-CD, indicating a limit of detection of approximately 10 ppm.

FIG. 3A is a plot of fluorescence intensity vs. concentration of free PFOA (ppm) to compare solvent ionic strength by comparing response of the reaction mixture containing water compared to 0.5×PBS (~60 mM NaCl, pH 7.0) with 10% (v/v) sample. A concentration of 0.5 mM p-β-CD was used with 0.008 mM ANS.

FIG. 3B is a plot of fluorescence intensity vs. concentration of free PFOA (ppm) to compare fluorescence response using ANS and p-β-CD with different buffers providing different pH ranges, in comparison to the 10% (v/v) water sample.

FIG. 3C is a plot of fluorescence intensity vs. concentration of free PFOA (ppm) to optimize the amount of sample input into the reaction mixture comprising 10%, 20% and 50% sample volume with 0.008 mM ANS.

FIG. 3D is a plot of fluorescence intensity vs. concentration of free PFOA (ppm) to compare the limit of detection with different concentrations of p-β-CD in water at 0.008 mM ANS.

FIG. 4A is a plot of normalized fluorescence signal vs. concentration of PFOA (ppm) to compare the signal response for p-β-CD with m-β-CD using 0.25 mM cyclodextrin and 0.008 ANS.

FIG. 4B is a plot of normalized fluorescence signal vs. concentration of PFOA (ppm) to compare the signal response for p-β-CD with 2-HP-β-CD using 0.25 mM cyclodextrin and 0.008 ANS.

FIG. 4C is a plot of normalized fluorescence signal vs. concentration of PFOA (ppm) to compare the signal response for p-β-CD with TAc-β-CD using 0.25 mM cyclodextrin and 0.008 ANS.

FIG. 5 is a plot of fluorescence intensity vs. concentration of PFOA (ppm) for four different PFAS samples including PFOS, PFOA, PFHxA and AFFF.

FIG. 6A is a plot of normalized fluorescence signal vs. concentration (ppm) of octanoic acid and PFOA in two replicate experiments to compare the specificity of detection of PFOA with the detection of PFOA using 0.25 mM p-β-CD and 0.008 ANS in water.

FIG. 6B is a plot of normalized fluorescence signal vs. concentration (ppm) of humic acid and PFOA in two replicate experiments to compare the specificity of detection of PFOA with the detection of humic acid using 0.25 mM p-β-CD and 0.008 ANS in water.

FIG. 7A is a plot of fluorescence intensity vs. concentration of AFFF (ppm) to compare the signal response using HPLC methanol grade of methanol conditioned through a WAX column.

FIG. 7B is a plot of reported concentration (ppb) vs. input concentration of PFOA (ppb) calculated by diluting PFOA stock solution into milliQ™ water.

FIG. 8 is a plot of normalized fluorescence signal vs. concentration of PFOA to indicate a prophetic response based on the data of FIG. 5. The signal was normalized and a concentration factor of 3,066× was applied to calculate the conversion between raw signal and PFOA concentration for FIG. 7B. The calculated concentration (Y axis) was plotted against the known concentration (X axis). The 3,066× factor was calculated based on the observed 15,333× concentration factor and the dilution required to add the p-β-CD & ANS mixture.

FIG. 9A is a cyclic voltammogram demonstrating p-β-CD forming an inclusion complex with para-aminophenol (PAP) at different concentrations of p-β-CD ranging between 5×10⁻⁴ mM to 5 mM.

FIG. 9B is a cyclic voltammogram demonstrating p-β-CD forming an inclusion complex with ferrocenemethanol (FcMeOH) at different concentrations of p-β-CD ranging between 0.016 mM and 64 mM.

FIG. 10A is a square wave voltammogram demonstrating p-β-CD forming an inclusion complex with ferrocenemethanol (FcMeOH) at different ratios of p-β-CD/FcMeOH in the presence of 20% methanol.

FIG. 10B is a square wave voltammogram demonstrating p-β-CD forming an inclusion complex with ferrocenemethanol (FcMeOH) at different ratios of p-β-CD/FcMeOH in the presence of 10% methanol.

FIG. 10C is a square wave voltammogram demonstrating p-β-CD forming an inclusion complex with ferrocenemethanol (FcMeOH) at different ratios of p-β-CD/FcMeOH in the absence of methanol.

FIG. 11A is a plot of current vs. concentration of free PFOA in an assay using different ratios of FcMeOH and β-cyclodextrin as determined by cyclic voltammetry.

FIG. 11B is a plot of the square wave current response vs. concentration of free PFOA in an assay using different ratios of FcMeOH and β-cyclodextrin as determined by square wave voltammetry.

FIG. 12 is a plot of fluorescence vs. concentration of monomeric α-cyclodextrin in an experiment to investigate any extent of binding of four indicator molecule candidates to monomeric α-cyclodextrin.

FIG. 13A is a plot of fluorescence vs. concentration of cyclodextrin in an experiment to investigate binding of the indicator molecule ANS to monomeric α-cyclodextrin and polymeric α-cyclodextrin.

FIG. 13B is a plot of fluorescence vs. concentration of cyclodextrin in an experiment to investigate binding of the indicator molecule bis-ANS to monomeric α-cyclodextrin and polymeric α-cyclodextrin.

FIG. 14 is a plot of normalized fluorescence signal vs. concentration of the PFAS molecule PFOA in an embodiment of an indicator displacement assay in the presence of 500 ppm octanoic acid as a model interferent to investigate the potential for monomeric α-cyclodextrin to sequester octanoic acid and improve the sensitivity of an embodiment of an indicator displacement assay.

FIG. 15 is a plot of normalized fluorescence signal vs. concentration of the PFAS molecule PFOA in an embodiment of an indicator displacement assay in the presence of 200,000 ppm benzene as a model interferent to investigate the potential for monomeric α-cyclodextrin to sequester benzene and improve the sensitivity of an embodiment of an indicator displacement assay.

FIG. 16 is a plot of normalized fluorescence signal vs. concentration of the PFAS molecule PFOA in an embodiment of an indicator displacement assay in the presence of 250 ppm sodium dodecyl sulfate (SDS) as a model interferent to investigate the potential for monomeric α-cyclodextrin to sequester SDS and improve the sensitivity of an embodiment of an indicator displacement assay.

FIG. 17A is a plot of relative fluorescence units vs. PFOA concentration in the presence of monomeric α-cyclodextrin and polymeric α-cyclodextrin at different concentrations in an embodiment of an indicator displacement assay in the presence of bis-ANS as an indicator molecule.

FIG. 17B is a plot of relative fluorescence units vs. SDS concentration in the presence of monomeric α-cyclodextrin (m-α-CD), and polymeric α-cyclodextrin (p-α-CD), at different concentrations in an embodiment of an indicator displacement assay in the presence of bis-ANS as an indicator molecule.

FIG. 18A is plot of relative fluorescence units vs. PFOA concentration in an embodiment of an indicator displacement assay conducted in the presence of monomeric α-cyclodextrin (m-α-CD), polymeric quaternary amine-modified β-cyclodextrin (pQ-β-CD) and with a combination thereof, using bis-ANS as an indicator molecule.

FIG. 18B is plot of relative fluorescence units vs. SDS concentration in an embodiment of an indicator displacement assay in the presence of monomeric α-cyclodextrin (m-α-CD), quaternary amine-modified β-cyclodextrin (pQ-β-CD) and with a combination thereof, using bis-ANS as an indicator molecule.

FIG. 19 is a plot of fluorescence vs. PFOA concentration in an embodiment of an indicator displacement assay in the presence of SDS and different concentrations of monomeric α-cyclodextrin (m-α-CD), and polymeric α-cyclodextrin (p-α-CD), using bis-ANS as an indicator molecule.

FIG. 20 is a plot of fluorescence vs. PFOA concentration in an embodiment of an indicator displacement assay conducted in the presence of different concentrations of SDS and different concentrations of monomeric α-cyclodextrin (m-α-CD), and polymeric α-cyclodextrin (p-α-CD), using bis-ANS as an indicator molecule.

FIG. 21 is a plot of reported PFAS concentration in ppb (parts per billion) determined using an embodiment of the indicator displacement assay as a function of the reported PFAS concentration determined using the conventional EPA 1633 method in ppb with the dotted line indicating alignment of the lowered concentrations determined when 45 mM of monomeric α-cyclodextrin are included in the multiple displacement assay.

FIG. 22A, Scheme A illustrates binding of an indicator molecule (IND) to p-β-cyclodextrin (p-β-CD) to provide a complex which provides elevated fluorescence relative to the indicator molecule in aqueous solution. Scheme B illustrates displacement of the indicator molecule from the complex by a PFAS molecule.

FIG. 22B, Scheme C illustrates competitive binding of an interferent (INT) with the indicator molecule (IND) for binding to p-β-cyclodextrin (p-β-CD), resulting in reduced fluorescence which affects the indicator displacement assay. Scheme D illustrates inclusion of monomeric α-cyclodextrin (m-α-CD) to sequester the interferent (INT) without affecting binding of the indicator molecule to p-β-cyclodextrin (p-β-CD), thereby rescuing the elevated fluorescence required for an accurate indicator displacement assay in Scheme D.

FIG. 23 is a plot of normalized fluorescence signal vs. PFAS concentration for p-β-cyclodextrin (p-β-CD) compared with polymeric quaternary amine modified β-cyclodextrin (PQ-β-CD).

FIG. 24A is a plot of normalized fluorescence signal vs. PFOA concentration in an experiment conducted to determine the effect of pre-treatment of a first environmental sample with a carbon column to remove interferents.

FIG. 24B is a plot of normalized fluorescence signal vs. PFOA concentration in an experiment conducted to determine the effect of pre-treatment of a first environmental sample with a carbon column to remove interferents.

FIG. 25 is a plot of fluorescence signal response (in relative fluorescence units) vs. PFOA concentration in an experiment conducted to determine the effect of different wash solutions for washing PFAS samples bound to a hydrophobic styrene divinyl benzene (SDVB) column.

FIG. 26A is a plot of fluorescence signal response (in relative fluorescence units) vs. PFOA concentration in an experiment conducted to determine the effect of combining the carbon column pre-treatment with methanol washing of the sample on a hydrophobic SDVB column for a first sample.

FIG. 26B is a plot of fluorescence signal response (in relative fluorescence units) vs. PFOA concentration in an experiment conducted to determine the effect of combining the carbon column pre-treatment with methanol washing of the sample on a hydrophobic SDVB column for a second sample.

FIG. 27A is a plot of normalized fluorescence signal vs. PFAS concentration showing a comparison of the effect of solid phase extraction (SPE) pre-treatment on accurate detection of PFAS for a first set of three different samples with either WAX or hydrophobic SDVB, using a standard PFAS curve for the assessment.

FIG. 27B is a plot of normalized fluorescence signal vs. PFAS concentration showing a comparison of the effect of solid phase extraction (SPE) pre-treatment on accurate detection of PFAS for a second set of four different samples with either WAX or hydrophobic SDVB, using a standard PFAS curve for the assessment.

FIG. 28 is a bar chart showing the results of a comparison of background fluorescence noise for a series of PFAS samples pre-treated with SPE using WAX or hydrophobic SDVB.

FIG. 29 is a plot of fluorescence signal response in relative fluorescence units vs. PFAS concentration indicating the effect of detection of PFAS following drying of the assay mixture and the effect of refrigerated storage for 33 days.

FIG. 30 is a plot of fluorescence signal response in relative fluorescence units vs. PFAS concentration comparing the effect of refrigerated storage and room temperature storage.

FIG. 31 is a plot of fluorescence signal response in relative fluorescence units vs. PFAS concentration comparing the effect of drying the assay mixture and including PFOA in the assay mixture.

FIG. 32 is a plot of fluorescence signal response (in relative fluorescence units) vs. PFOA concentration (ppm) in an experiment conducted to determine the effect of the degree of substitution of p-β-cyclodextrin (p-β-CD) with a quaternary amine substituent. DS1.5 indicates an average of 1.5 quaternary amine substituents in each β-cyclodextrin unit of the p-β-CD. Likewise, DS3 indicates an average of 3 quaternary amine substituents in each β-cyclodextrin unit of the p-β-CD and DS4.5 indicates an average of 4.5 quaternary amine substituents in each β-cyclodextrin unit of the p-β-CD.

FIG. 33 is a plot of fluorescence signal response (in normalized fluorescence units) vs. concentration (ppm) of six different species of PFAS molecules including perfluorononanoic acid (PFNA), perfluorooctanoic acid (PFOA), perfluorooctane sulfonate (PFOS), perfluorohexane sulfonate (PFHxS), perfluorobutane sulfonate (PFBS), and hexafluoropropylene oxide dimer acid (HFPO-DA, also commonly known as GenX).

FIG. 34 is a plot of fluorescence signal response (in relative fluorescence units (RFU)) vs. concentration (ppm) of seven different species of PFAS molecules including perfluorononanoic acid (PFNA), perfluorooctanoic acid (PFOA), perfluorooctane sulfonate (PFOS), perfluorohexane sulfonate (PFHxS), perfluorobutane sulfonate (PFBS), hexafluoropropylene oxide dimer acid (HFPO-DA, also commonly known as GenX), and perfluorobutanoic acid (PFBA).

FIG. 35 is a plot of fluorescence signal response (in relative fluorescence units (RFU)) vs. concentration (ppm) of four different species of PFAS molecules including perfluorobutanoic acid (PFBA), perfluorooctanoic acid (PFOA), perfluorobutane sulfonate (PFBS), and perfluorotetradecanoic acid (PFTDeA).

FIG. 36 is a plot of fluorescence signal response (in relative fluorescence units (RFU)) vs. concentration (ppm) of two different species of PFAS molecules; perfluorooctanoic acid (PFOA), and perfluoropropanesulfonic acid (PFPrS).

FIG. 37 is a plot of fluorescence signal response (in relative fluorescence units (RFU)) vs. concentration (ppm) of two different species of PFAS molecules; perfluorooctanoic acid (PFOA), and 6:2-fluorotelomersulfonic acid (6:2 FTS).

### DETAILED DESCRIPTION

### Introduction and Rationale

This technology relates to the detection of PFAS compounds within environmental samples. PFAS compounds, one of the classes of “forever chemicals,” are toxic and recalcitrant. These compounds are characterized by the presence of fluorine atoms along a carbon backbone. They have many commercial uses such as waterproofing, fire suppression, manufacturing, and inclusion in various products as stability additives. They enter the environment from multiple sources, including through the breakdown of PFAS-containing products over time (i.e., in landfills), through runoff from manufacturing processes, and through the cleaning after fire suppression events at airports, military bases, and ports.

The detection of PFAS compounds in the laboratory is cumbersome and labour intensive. Detection in the field at the location of contamination is currently non-existent. Conventional monitoring methods used in analytical labs rely on LC-MS/MS or combustion ion chromatography. These are expensive, time-consuming, require highly trained personnel, and rely on significant infrastructure investments. Current turnaround times for PFAS lab testing runs between 3 and 16 weeks depending on the exact test performed, and costs hundreds, sometimes thousands, of dollars per test. Rush testing is available with results available in one week, but this is very expensive. While testing is in progress, companies often attempt to remediate PFAS contamination blindly and are required to pay for onsite personnel and to store large volumes of water contaminated with PFAS that may or may not be at a concentration where it can be discharged.

The technology described herein uses advances in sample preparation and an assay developed using a cyclodextrin molecule and an indicator molecule in a field-deployable assay system in combination with a raw sample concentrating process which can detect PFAS molecules at low concentrations.

A recent review of sensors for detecting PFAS compounds has recognized that cyclodextrins have been used for adsorption and removal of PFAS compounds to remediate water sources but have not yet been developed and evaluated as a sensor for detecting PFAS compounds (Menger et al., Chem. Eng. J., 2021, 417, 129133).

The present inventors have been engaged in development of field deployable electrochemical detectors for analyzing environmental samples for various analytes (see for example, U.S. Pat. Nos. 9,689,046 and 10,415,102, which are each incorporated herein by reference in entirety). During research in development other assays, modifications of β-cyclodextrin to increase affinity for PFAS molecules were considered and examined. It was surprisingly discovered that certain modifications to β-cyclodextrin produced higher affinity for a group of PFAS molecules relative to unmodified β-cyclodextrin, thereby providing advantageous limits of detection and high reproducibility. The inventors sought to explore if these differences were a result of the cyclodextrin-PFAS interaction or a result of some other interaction in the assay by utilizing an alternate assay that specifically addresses cyclodextrin guest-host interactions. This led to the surprising discovery that an indicator displacement assay based on displacement of a cyclodextrin-bound indicator molecule by a PFAS compound could serve as the basis for a sensitive PFAS sensor capable of quantification of common PFAS molecules, or mixtures thereof. It was then recognized by the present inventors that such an assay could be developed and commercialized at relatively low cost and that it could be readily configured for use with a field deployable analysis system that could provide rapid PFAS detection and quantification results to guide and improve the efficiency of remediation efforts. Embodiments of the indicator displacement assay described herein do not distinguish between different PFAS molecules but provides a process for quantifying one PFAS compound, if only one was present and would also provide a process for quantifying a total amount of all PFAS compounds in a given sample if multiple PFAS molecules were present.

### PFAS Compounds

The United States Environmental Protection Agency (EPA) curates a master list of PFAS compounds on its internet site currently at comptox.epa.gov. Some of the more common PFAS perfluorononanoic acid (PFNA), perfluorooctanoic acid (PFOA), compounds include perfluorooctane sulfonate (PFOS), perfluorohexane sulfonate (PFHxS), perfluorobutane sulfonate (PFBS), perfluorobutanoic acid (PFBA), perfluorotetradecanoic acid (PFTDeA), perfluoropropanesulfonic acid (PFPrS), 6:2 fluorotelomersulfonic acid (6:2 FTS) 8:2-fluorotelomersulfonic acid (8:2 FTS), perfluoro-3,6-dioxaheptanoic acid (NFDHA), perfluorodecanoic acid (PFDA), perfluoroheptanoic acid (PFHpA), perfluoroheptanesulfonic acid (PFHpS), perfluoro-4-methyoxybutanoic acid (PFMBA), perfluoro-3-methoxypropanoic acid (PFMPA), perfluoropentanoic acid (PFPeA), trifluoroacetic acid (TFA), perfluoroundecanoic acid (PFUnA), and hexafluoropropylene oxide dimer acid (HFPO-DA, also commonly known as GenX). Notably, each of these compounds includes at least one ionizable functional group which will be protonated or deprotonated, depending on the pH of the solution and the pKa value of each individual ionizable group. For the sake of simplicity, the compound names listed above are intended herein to refer to both the protonated and deprotonated forms as well as salts thereof.

### Cyclodextrins and Derivatives Thereof

Cyclodextrins are a family of cyclic oligosaccharides, consisting of a macrocyclic ring of glucose subunits joined by α-1,4 glycosidic bonds. Cyclodextrins are produced from starch by enzymatic conversion and are used as additives in food, pharmaceutical, drug delivery, and chemical industries, as well as agriculture and environmental engineering. Cyclodextrins are composed of 5 or more α-D-glucopyranoside units linked 1->4, as in amylose (a fragment of starch). Typical cyclodextrins contain a number of glucose monomers ranging from six to eight units in a ring, creating a cone shape. Common examples include α-(alpha)-cyclodextrin, which has 6 glucose subunits, β-(beta)-cyclodextrin, which has 7 glucose subunits, and γ-(gamma)-cyclodextrin, which has 8 glucose subunits. The cyclodextrins have toroidal shapes, with the larger and the smaller openings of the toroid exposing to the solvent secondary and primary hydroxyl groups respectively. Because of this arrangement, the interior of the toroids is not completely hydrophobic, but considerably less hydrophilic than the aqueous environment and thus able to host other hydrophobic molecules, thereby forming what is known as an inclusion complex or a host-guest complex. In contrast, the exterior is sufficiently hydrophilic to provide the cyclodextrins with solubility in water and other polar solvents.

Without being bound by any particular theory, the inventors postulate that a cyclodextrin with high affinity for PFAS compounds may be provided when the cyclodextrin is provided with one or more functional group substituents which can interact with the head group of a given PFAS compound (for example —COOH or —SO₃ if PFOA or PFOS, respectively) or with the backbone (i.e., —CF₂-units). It is assumed that a negatively-charged PFAS functional group can be stabilized within a cyclodextrin which includes substituents having these features. Negatively charged PFAS functional groups are generally more common among PFAS molecules. However, PFAS molecules having positively charged or functional groups or functional groups with partial positive charges would be stabilized within a cyclodextrin having different substituents which provide electron donating effects such as alkyl groups, substituents with resonance donating structures, and substituents having lone pairs of electrons.

In one embodiment, there is provided a cyclodextrin modified with a long-CF₂-tail on one end of the toroid of the cyclodextrin to allow the cyclodextrin to interact with long chain PFAS (for example, greater than ten carbons in length, such as perfluorododecanoic acid, which is 12 carbons long). In other embodiments, cyclodextrins having one or more functional group substituents such as primary amine (—NH₂), protonated primary amine (—NH₃⁺), protonated secondary amine (—NRH₂⁺), protonated tertiary amine (—NR₂H⁺), or quaternary amine (—NR₃⁺), promote interactions with the negatively charged acid or sulfonyl groups of the PFAS molecule. Other substituents which are reasonably predicted to provide the cyclodextrin with similar favorable properties for an indicator displacement assay are electron withdrawing groups which may be formally neutral while having a partial positive charge/dipole or positively charged substituents including, but not limited to: cyano, azido, nitro, hydroxypropyl, hydroxyamino, aldehyde, carboxyl, acetyl, ester, halogen, haloalkyl, hydroxyl, alkoxyl, and sulfonyl.

In other embodiments, where the cyclodextrin is in polymeric form, the monomer units are connected by linkers which include functional groups that can increase affinity for PFAS compounds. Examples of such linker modifications are described in Wang et al., ACS Central Science, 2022, 8, 663, which is incorporated herein by reference in its entirety.

PFAS compounds may be classified into subsets according to their chemical properties. There is currently a strong need to detect compounds such as PFOA and PFOS, which are negatively charged at neutral pH. It is expected that detection of positively charged PFAS compounds will become important as well, as this subset of PFAS is newer and less well regulated. A cyclodextrin with anionic functional groups to provide a negatively charged cyclodextrin could be used to improve affinity for positively charged PFAS compounds. For detection of zwitterionic PFAS compounds, the inventors have conceived of an embodiment involving designing a cyclodextrin with suitable spacing of carboxylic acid and amino groups to increase the affinity of the modified cyclodextrin for a given zwitterionic PFAS compound, to lower the detection limit of an indicator displacement assay. In another embodiment, a pair of cyclodextrins could be used to create a sensor for cationic and anionic PFAS by having respective carboxylic and amino substituents on their outer surfaces such that the amino-modified cyclodextrin is positively charged at pH 7 while the carboxyl-modified cyclodextrin is negatively charged at pH 7. This combination of cyclodextrins will be useful for detecting zwitterionic PFAS compounds in the indicator displacement assay. While some of these modified cyclodextrins are known in the art, they are described in terms of absorption and remediation and have not been incorporated into an assay to use as a sensor for quantification of PFAS compounds. It is to be understood that such modifications may be applied to cyclodextrin monomers and polymers.

### Indicator Molecules

The present technology employs indicator molecules to provide a measurable parameter which indicates binding of a PFAS compound. In this context, “binding” refers to entry of another molecule into the cavity of a cyclodextrin in a host-guest interaction, which is presumed to be dominated by hydrophobic interactions and other non-covalent interactions such as inductive effects and steric effects. The parameter provided by the indicator molecule may be any measurable physical parameter. Examples of such parameters of indicator molecules include, but are not limited to, color change (colorimetric analysis), fluorescence, and change in oxidation state (redox chemistry). Indicator molecules for colorimetric analysis and fluorescence detection include dye molecules (also known as chromophores or fluorophores), which have conjugated multi-ring structures. Some examples of fluorescence indicator dyes include, but are not limited to, cyanines such as Cy3-COOH) and Cy5-COOH), xanthenes such as fluorescein and rhodamine, pyrenes such as 8-hydroxypyrene-1,3,6-trisulfonic acid, trisodium salt (HPTS) and 6,8-dihydroxy-1,3-pyrenedisulfonic acid (DHPDS), napthalenes such as 8-anilinonaphthalene-1-sulfonic acid (ANS), bis-ANS, Acid Blue and Acid Black, and other fluorescence indicators such as SYPRO™ Orange (Thermo Fisher (S6650), BODIPY-FL, 7-nitrobenz-2-oxa-1, 3-diazole-4-yl, naphthalimide (lucifer yellow), and acridine orange. Colorimetric indicator molecules are also used in the indicator displacement assays. Examples of such colorimetric molecules include dyes such as Acid Blue 120 and Acid Black 24. Indicator molecules for redox detection are also known as electroactive analytes. Examples of such electroactive analytes are described in U.S. Pat. Nos. 9,689,046 and 10,415,102. Additional examples include metallocenes containing metals such as iron, cobalt, chromium, nickel ruthenium and vanadium. Ferrocene and derivatives such as ferrocenemethanol, ferrocenedimethanol, ferrocenecarboxaldehyde, ferrocenecarboxylic acid, and aminoferrocene are expected to be useful as electroactive compounds in the indicator displacement assays described herein. Such compounds are capable of being reduced and/or oxidized when a voltage is applied to an electrical cell and provide a measurable electrical current in the electrical cell. Other examples of indicator molecules may be bifunctional by providing a chromophore while being electroactive. Some embodiments may include a mixture of indicators to provide a visual quality control metric to a user while a more quantifiable result is produced electrochemically using an electroactive indicator molecule.

### Indicator Displacement Assays

An indicator displacement assay is based on a supramolecular assembly of an indicator molecule that is reversibly bound to a host molecule. In the presence of an analyte, the indicator molecule is displaced from the host, resulting in a measurable change in a physical parameter. The target analyte must also have a higher affinity than the affinity of the indicator molecule at a particular concentration. The term “affinity” refers to the extent that the target analyte is bound relative to the indicator molecule, and thus the affinity is related to the association constant (sometimes referred to as the “formation constant”) and concentration of the host, guest, and indicator molecule, to achieve an effective displacement of the indicator and provide a measurable change of a parameter (Sedgewick et al. Chem. Soc. Rev., 2021, 50, 9). The change of the parameter will indicate the amount(s) of the target analyte(s) in the sample being assayed.

### Kits Including Containers for Indicator Displacement Assay Components

Another embodiment provides kits which include containers which are formed of materials such as plastics which are substantially free of PFAS compounds, in order to avoid assay interference. In one embodiment, separate containers separately containing cyclodextrin and an indicator molecule are provided. In a preferred embodiment, the cyclodextrin is provided in a mixture with an indicator molecule such that an extra step of mixing the cyclodextrin with the indicator is not required at the point of analysis. The mixture may be provided in a single container in either solution form or lyophilized form. A lyophilized form would provide a way to further increase the concentration of the PFAS sample being analyzed. The kits are configured to facilitate conducting indicator displacement assays in the field where environmental samples such as water samples requiring analysis for the presence of PFAS compounds.

The cyclodextrin of the kit may be any one of the cyclodextrins described herein, including but not limited to m-β-CD, p-β-CD, 2HP-β-CD, TAc-β-CD, 6-amino-β-CD, and any derivative or polymer (including polymers with various linkers describe herein) thereof, which have suitable affinity for an indicator molecule. The indicator molecule of the kit may be a colorimetric indicator molecule, a fluorescence indicator molecule, or an electroactive molecule. The indicator molecule may be any one of the indicator molecules described herein, including but not limited to the fluorescence indicator Nile Red or ANS, or the electroactive indicators PAP, ferrocene or ferrocenemethanol.

The kit may include an empty container which is formed of a material such as a plastic which is substantially free of PFAS compounds, which is provided for the purpose of collecting a water sample for analysis to detect and quantify the presence of one or more PFAS compounds.

The kit may further include other components to generate a concentrated version of a collected environmental sample. Such additional components may include a resin such as a weak anion exchange (WAX) resin, which may be provided in a pre-packaged column such as an Agilent Bond Elut PFAS WAX column, which is capable of binding PFAS compounds while eluting other common environmental contaminants. One or more syringes may be provided with the kit to facilitate transfer of the sample to the chromatography column. The kit may also include an evaporator to remove methanol from an eluted sample suspected of containing one or more PFAS compounds, to allow the PFAS compounds to be resuspended in another solvent at a reduced volume, thereby raising the concentration of the PFAS compounds for subsequent detection. The evaporator may be in the form of a heating block, a vacuum line, a line to provide a stream of dry gas, or any other device configured to evaporate a solvent such as water and/or other polar or nonpolar solvents from a sample.

### Interferents of PFAS-Contaminated Sites

Sites known to be contaminated with significant concentrations PFAS, such as sites where aqueous film-forming foams (AFFFs) have been used, are being tested to determine the extent of PFAS contamination and the related effectiveness of remediation efforts being conducted at these sites. Examples of such sites include firefighting training areas or sites of industrial fires where significant volumes of AFFFs have been released into the environment. As a result, the PFAS-contaminated sites also include significant amounts of various surfactants which may have been included in the AFFF formulations or used in previous remediation efforts or present in the environment prior to the release of the AFFFs. A common remediation strategy is a process known as foam fractionation which involves separation of organic compounds, colloidal particles and suspended solids from aqueous solution using a rising stream of bubbles and may include detergents as surface-active agents to increase the efficacy of foam fractionation processes. The inventors recognized that common surfactants based on alkyl groups with lengths of 4 or more carbons, would be likely to bind to cyclodextrins and interfere with embodiments of the indicator displacement assay described herein by reducing the range of fluorescence provided by the complex of the indicator molecule with the cyclodextrin used in the indicator displacement assay. As a result, the inventors recognized that it would be advantageous to modify the assay to include a scavenger molecule which selectively binds hydrocarbons, and/or surfactants having alkyl chains with 4 or more carbons and which does not significantly bind the indicator molecule, and which does not significantly bind PFAS compounds. An example of such a surfactant expected to be present at significant concentrations at PFAS-contaminated sites is sodium dodecyl sulfate (SDS). Other structurally similar surfactants may include, but are not limited to: lauryldimethylamine N-oxide (LDAO); 3-laurylamido-N,N″-dimethylpropylaminoxide (LAPAO) dodecylphosphocholine (DPC, also known as Foscholine-12 (FC12)), β-octyl glucoside (β-OG), dodecyl maltoside (DDM) and members of the DOWFAX™ family of ionic surfactants manufactured by Dow Chemical/Dupont. Since these detergents have alkyl chains at least 6 carbons in length, they are more likely to interfere with cyclodextrin-based displacement assays than compounds which include aromatic ring structures, which are generally too wide in dimension to bind to common cyclodextrins. Therefore, interferent compounds are co-contaminating compounds which may include, for example, hydrocarbons, and surfactants having alkyl chains at least 6 carbons in length. Such interferent compounds are currently considered among the most concerning of interferents with respect to assay interference. However, as used herein, the term “interferent” refers to any substance whose presence interferes with an analytical procedure via any mechanism. Although many other hydrocarbons such as benzene, toluene, xylene, polychlorinated biphenyls, dioxins and furans are typically present at PFAS-contaminated sites, such compounds are less likely to interfere with cyclodextrin-based displacement assays, due to the sizes of the ring structures of these compounds. However, the possibility remains that certain examples of such compounds may function as interferents by causing other issues with the cyclodextrin-based displacement assays, such as agglomeration and precipitation of assay components, for example through non-specific interactions or through specific chemical reactions which could occur between the interferents and assay components.

Examples 8 to 17 below describe the result of experiments performed to develop an interferent scavenger to prevent interference with a PFAS detection assay based on displacement of an indicator molecule from a cyclodextrin. As used herein, the term “scavenger” refers to any chemical compound included in an assay for the purpose of sequestering an interferent which would otherwise interfere with the assay. It is to be understood that while the example scavengers described herein are based on α-cyclodextrins, which are investigated in terms of sequestering long chain hydrocarbons and surfactants, if a given PFAS-contaminated site includes another class of interferents which are found to interfere with the assay, an appropriate scavenger appropriate for that class of interferents can be identified and adapted for use in the assay without undue experimentation.

### Removal of Non-Specific Interferents by Pre-Treatment of Samples

An aspect of this technology is allowing the assay to be performed in the field, and to function adequately in environmental samples which will include many additional compounds in addition to the PFAS target molecules. As noted above, at least some co-contaminating compounds are expected to act as interferents.

It will be appreciated that the most controlled environment is a laboratory setting, where chemicals are stored as methanolic solutions in freezers or in flammable cabinets. Alternatively, there are analytical labs that can detect PFAS in environmental samples, but that requires off-site shipping.

Current systems in place for PFAS monitoring rely on analytical laboratory methodologies. EPA methods 537.1 and 1633 are LC-MS/MS methods that can quantitate PFAS compounds in environmental samples. These technologies first extract PFAS from a sample utilizing solid phase extraction columns, followed by elution, then evaporation, and then resuspension as a concentrated solution that is then injected into a LC-MS/MS system. This provides exceptional sensitivity but is slow and most analytical labs are experiencing significant backlogs for analysis of environmental samples being analyzed for the presence of PFAS compounds.

The inventors have identified that EPA methods 537.1 and 1633 may not always function adequately for some environmental samples. The inventors identified that at least some of the issues with the EPA methods related to the presence of interferents which negatively alter the response of the assay.

To isolate the target of interest, the present technology may include one or more of the following adaptations:
The utilization of carbon adsorbents prior to sample extraction to remove organic contaminants that may interfere with the indicator displacement assay or the binding of PFAS molecules to the solid phase extraction column.
The utilization of dilute methanolic rinses to the adsorbed PFAS on a solid phase extraction column to remove interferents that were co-adsorbed.
The utilization of alternate solid phase extraction columns such as weak anion exchange (WAX), or hydrophobic columns using hydrophobic resins presenting hydrophobic moieties such as styrene divinyl benzene (SDVB), for example, to provide different mechanisms to capture PFAS molecules and co-adsorbents in different sample matrices.

For example, PFAS-containing samples may be treated by foam fractionation with the objective of creating a concentrated PFAS-containing “foamate” layer and a PFAS-free layer. It is advantageous to have the capacity to analyze such foamate layers using embodiments of the cyclodextrin-based indicator displacement assay described herein. It was found that weak anionic exchange (WAX) columns provided the best IDA functionality, as evaluated by the quantification of PFAS from the IDA compared to the gold-standard LC-MS/MS, but in groundwater samples it was found that SDVB (styrene divinyl benzene, a hydrophobic resin) was found to be a more effective column resin for removal of interferents. These are particularly surprising results because the conventional laboratory LC-MS/MS procedures do not make such distinctions. Therefore, according to one embodiment, if the environmental sample is a foamate sample, the sample is subjected to a pre-treatment step comprising elution of the sample from a WAX column. According to another embodiment, if the environmental sample is a groundwater sample, the sample is subjected to a pre-treatment step comprising elution of the sample from a hydrophobic column.

In some more varied sample matrices (e.g., landfill leachate), there were examples where sample pre-treatment steps using WAX columns provided more favorable results and other examples where the sample pre-treatment steps using the SDVB column provided more favorable results. As used herein, the term “leachate” refers to water that has percolated through a solid matrix (such as soil and landfill materials, for example) and leached out some of the constituent components present in the matrix. It is to be understood that if the most common interferents present in the leachate are known, this information may be used to guide the selection of an appropriate sample pre-treatment step.

### Storage and Transport of IDA Components

As noted above, the components of the cyclodextrin-based indicator displacement assay include a cyclodextrin an indicator molecule and optionally an interferent scavenger compound. Control/calibration samples comprising known concentrations of PFAS compounds are also used. In one embodiment a premade mixture containing the main assay cyclodextrin, the indicator molecule and optionally the interferent scavenger compound is provided. It was established that this premade mixture can be stored in both a dry or a liquid format, as well as at room temperature or at 4 °C with room temperature being preferable to avoid cold chain requirements during shipment.

In some embodiments, the premade mixtures include PFAS compounds for controls and calibration. Since the PFAS compounds are in methanolic solutions, the shipment of liquid formats presents a challenge due to the flammable nature of the methanol, which makes a dried format preferable in some situations. Unexpectedly, the optimal configuration for storage of a premade mixture in a dried format includes both the assay components and the PFAS standards together prior to drying in order to create optimal results. Without being bound by any particular theory, it is believed that the optimal results are enhanced by an unexpected rehydration artifact.

### Field-Operable Apparatus for Performing the Indicator Displacement Assay

Embodiments of the technology described herein are adapted for implementation using a field-operable apparatus. Of note, SDVB and WAX columns require specific flow rates across the columns in order to ensure adequate contact time and proper binding of the PFAS compounds. The inventors have identified embodiments that enable evaporation of column eluents in a manner similar to the procedures of EPA 1633. Various embodiments of a field operable apparatus may include peristaltic pumps to control column elution flow rates, scaffolding to hold columns, resistive heating elements to maintain temperatures and evaporate eluents, as well as vacuum systems to apply negative pressure and increase eluent evaporation speed. These embodiments may be powered using alternating current or battery packs, the latter which is expected to provide a significant advantage for field analysis. Embodiments of the field-operable apparatus represent a dramatic improvement over standard laboratory practices that require bulky vacuum manifolds, ovens, and/or dedicated and automated solid phase extraction equipment.

### EXAMPLES

Example 1: Investigation of Fluorescent Dyes as Indicator Molecules for Binding of PFAS Compounds to a Cyclodextrin—It is known that numerous molecules can interact with cyclodextrins for an indicator assay, but for quantification of a particular molecule such as a PFAS compound, it is essential for the binding interaction to provide a measurable parameter which reflects the binding process in a reproducible manner. Polarity-sensitive fluorescent dyes change in intensity when in either an aqueous or a hydrophobic environment. The interior of the cyclodextrin is hydrophobic so it was postulated that binding of a fluorescent dye to a cyclodextrin should produce an increase in fluorescence relative to its free presence in aqueous solution.

Two fluorophore dyes were investigated for this purpose: Nile Red (9-(diethylamino)-5H-benzo[a]phenoxazin-5-one) and 8-anilinonaphthalene-1-sulfonic acid (ANS). For both ANS and Nile Red, the fluorescence intensity increases when the dye is contained within the β-cyclodextrin. Four different β-cyclodextrins were examined in this experiment; unmodified β-cyclodextrin (Sigma-Aldrich C4767) (also designated β-CD or m-β-CD to indicate monomeric β-CD to distinguish from polymeric-cyclodextrin which is described below); 2-hydroxypropyl-β-cyclodextrin (Sigma-Aldrich H107) (2HP-β-CD) (with a variable degree of substitution ranging from 0.5-1.3 units of 2-hydroxypropyl per glucose unit); triacetyl-β-cyclodextrin (Sigma-Aldrich 332623, or TCI T1844) (TAc-β-CD) (all hydrogen substituents replaced with an acetyl group); and β-cyclodextrin polymer (Sigma-Aldrich C2485) (also referred to herein as polymeric β-cyclodextrin and p-β-CD). This β-cyclodextrin polymer has an oxacyclopropane (oxirane) linking group linking the cyclodextrin glucose monomers at the C-6 position as shown in the structure below.

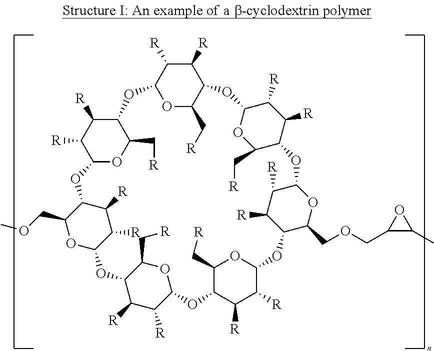

Linkers other than oxacyclopropane may be used in alternative polymers in additional embodiments. Examples of alternative linkers include, but are not limited to C2 to C6 substituted or unsubstituted alkyl chains, or other cyclic ether linkers such as oxacyclopropane, oxacyclobutane, oxacyclopentane, oxacyclohexane and furan, for example. Additional linkers are described in Wang et al., ACS Central Science, 2022, 8, 663, and Liu et al. Prog. Polymer Sci., 2021, 118, 101408, which are each incorporated herein by reference in entirety.

Another modified β-cyclodextrin which was identified as potentially having high affinity for PFAS compounds, but not yet tested is 6-amino-β-CD, which has an amino substitution at the 6-position of each glucose monomer of the β-cyclodextrin (Shinsei Chemical Company, SCHEM03466).

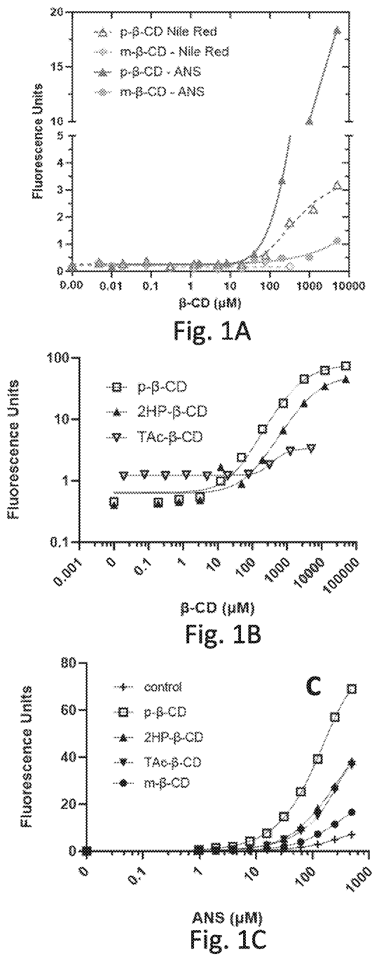

The results of the guest-host interactions between different cyclodextrins and fluorescence indicator dyes are shown in FIGS. 1A, 1B and 1C, where fluorescence was measured in a fluorescence plate reader with ANS measured with excitation of 375 nm and emission at 477 nm. Nile red was measured with excitation of 533 nm and emission at 637 nm. For both dyes, successful binding was surprisingly observed with polymeric-β-cyclodextrin but not the unmodified monomeric version and with varying success for the two modified monomers 2-hydroxypropyl-β-CD and triacetyl-β-CD (FIG. 1A). As the intensity of fluorescence and affinity observed for Nile Red were found to be generally lower (FIG. 1A), the ANS indicator molecule was selected for further experiments.

Characterization of the binding of ANS to the different β-cyclodextrins required having a dilution series of the cyclodextrin in the presence of the ANS dye indicator molecule (FIG. 1B). This was only achievable with the polymeric, 2-hydroxypropyl and triacetyl modified β-cyclodextrin, as the monomeric β-cyclodextrin was found to be insoluble at the concentrations required. Of the three modified β-cyclodextrins tested, it was surprisingly found that polymeric β-cyclodextrin demonstrated the strongest interaction with ANS. A concentration of 0.25 mM of each cyclodextrin was combined with a dilution series of ANS against a control sample without any β-cyclodextrin. The results shown in FIG. 1C indicate that polymeric β-cyclodextrin has the highest affinity (as based on the lowest concentration of dye needed to produce increases in fluorescence and demonstrating a linear increase in intensity). The two modified monomers, 2-hydroxypropyl-β-cyclodextrin and triacetyl-β-cyclodextrin, were found to have equal and lower affinities for ANS, while the monomeric unmodified β-cyclodextrin was found to have the least affinity, with a very small difference in ANS fluorescence compared to its increased intensity without cyclodextrin.

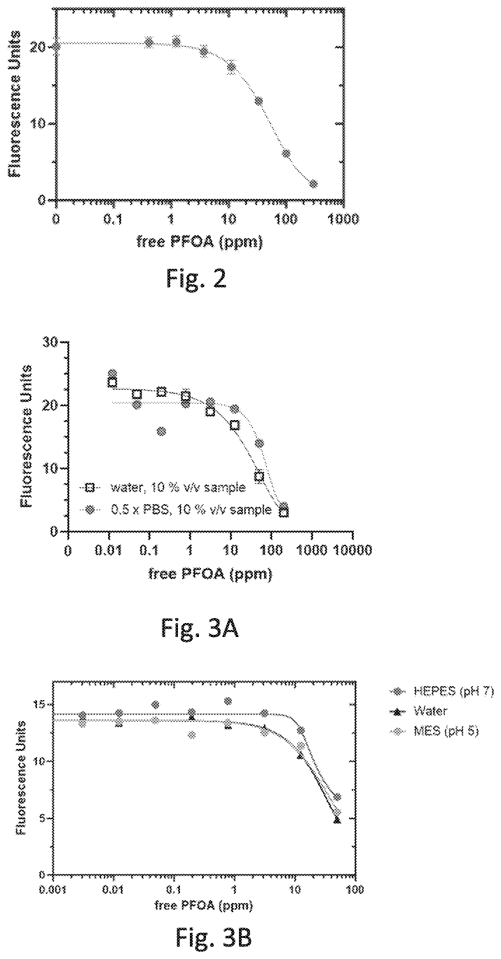

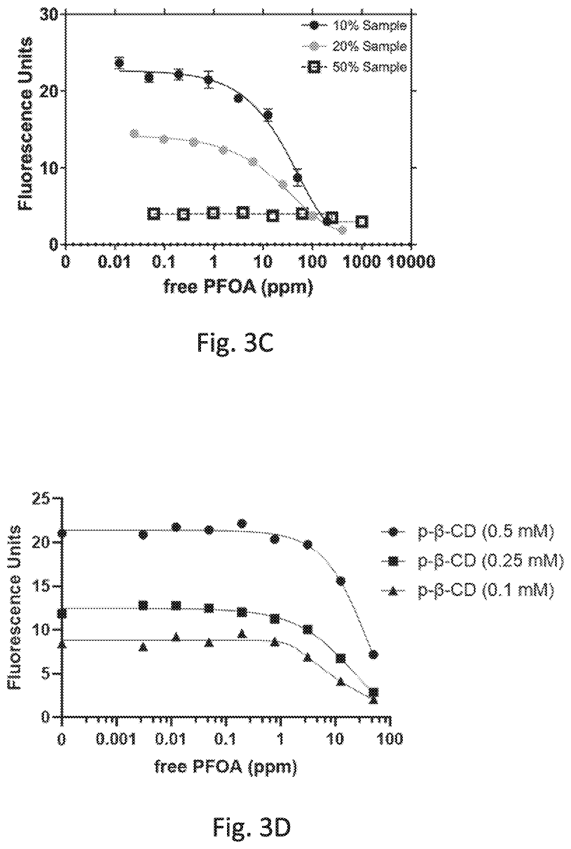

The inventors had discovered that perfluorooctanoic acid (PFOA) competes with other molecules for cyclodextrins, making it feasible to add PFAS molecules to compete with the fluorescent indicator from the cyclodextrin. By adding increasing amounts of a PFAS compound, a shift of the binding equilibrium in favor of the PFAS-β-cyclodextrin interaction was generated, thereby providing the basis for a sensitive assay. The preliminary results for the indicator displacement assay are shown in FIG. 2, and show a limit of detection of PFOA of about 10 ppm using the polymeric β-cyclodextrin and ANS.
Example 2: Optimization of the Indicator Displacement Assay—With the finding that the assay could be performed with appropriate sensitivity, several experiments were performed to characterize and optimize the assay for varying field conditions and to lower the limit of detection. Different optimization experiments for the ionic strength of the solution (FIG. 3A), the pH (FIG. 3B), the amount of methanolic sample input (FIG. 3C), and different concentrations of p-β-cyclodextrin were performed (FIG. 3D).

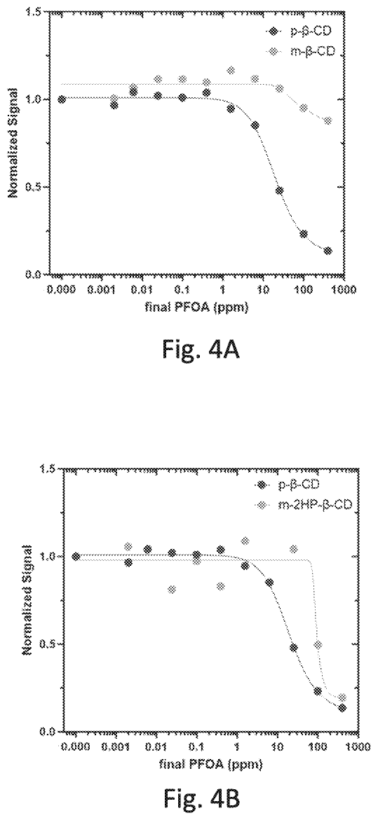

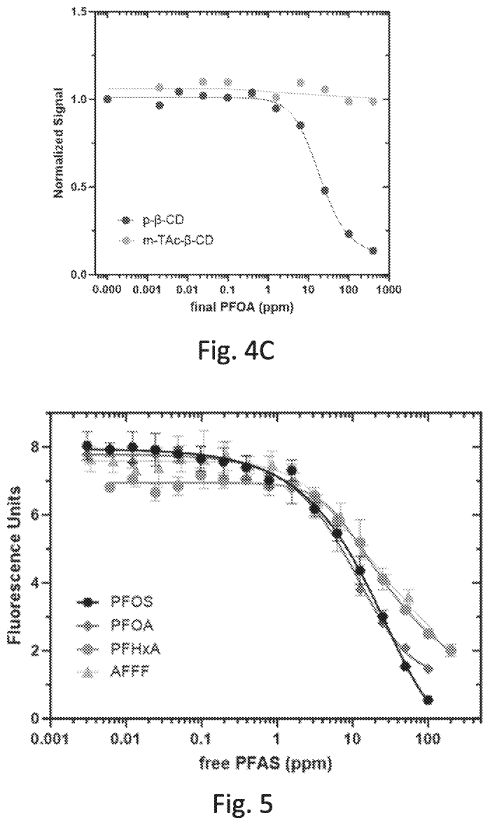

The results of these experiments suggest the use of buffer-free water to provide low ionic strength and higher sample content in solution to reduce the dilution of the target, and a β-cyclodextrin concentration of 0.25 mM. The ANS concentration was retained at a constant level of 0.008 mM. As the polymeric β-cyclodextrin produced the most robust and reproducible response with optimized conditions, the responses of the other cyclodextrins to PFOA using these optimized conditions were examined. The results are shown in FIGS. 4A-C. While a PFOA concentration-dependent change was observed with polymeric cyclodextrin, the other cyclodextrins show very small fluorescence responses and little change with the concentration of PFOA.
Example 3: Analysis of Additional PFAS Compounds—To increase the understanding of the limitations and specificity of the indicator displacement assay, testing of different types of PFAS compounds was needed. Pure samples of polyfluorooctanoic acid (PFOA), perfluorooctanesulfonate (PFOS), perfluorohexanoic acid (PFHxA) were investigated, as well as a proprietary mixture of different PFAS compounds in a 3% aqueous film-forming foam (AFFF) (Chemguard C3B, Chemguard, Marinette, WI, USA), which is a specialized product to provide fire suppression for class B hydrocarbon fuel fires.

The responses for the different individual PFAS compounds and the AFFF product are similar and the limit of detection for each of these PFAS samples is presented in FIG. 5 in assays using polymeric β-cyclodextrin and ANS as the indicator molecule.

Aliquots of 2 mg/mL stock solution were prepared in 1 mL of HPLC grade methanol (for PFOA, PFHxA and AFFF) and 1 mg/mL stock solution were prepared in 1 mL of HPLC methanol (for PFOS) using glass vials. The stock solution was then used to prepare serial dilutions. After mixing and vortexing, each serial dilution was mixed 10 times with a pipette and 20 μL was pipetted out and added to PCR tubes containing 80 μL of the IDA mixture containing 0.3125 mM of p-β-CD and 10 UM of ANS to provide final concentrations of 0.25 mM of p-β-CD and 0.008 mM ANS in water. The solution was then mixed using a vortex mixer and incubated at room temperature for 2 hours in a box to prevent photobleaching. A 90 μL aliquot of each solution was then transferred into a black plate, and measured using a spectrophotometer with excitation of 375 nm and emission at 477 nm. Each experiment was repeated 3 times. The limit of detection was calculated (Ambruster et al., Clin. Biochem. Rev., 2008 29, S49), using LoB (limit of blanks), LoD (limit of detection) as follows:

LoB=mean(blank)−1.645(SD(blank))

LoD=LoB−1.645(SD(low concentration sample))

The equations are converted to subtractions due to the detection intensity response decreasing upon release of the indicator molecule from the cyclodextrin. The limit of detection data determined for the PFAS samples (average of three replicates) is PFOA: 1.56 ppm; PFOS: 1.56 ppm; PFHxA: 6.25 ppm and AFFF: 6.25 ppm. The ppm concentrations outlined above represent the concentration of PFAS in the reaction mixture and does not consider preconcentration and dilution steps that are used to manipulate the original sample. However, the concentration level of PFAS in the original may be readily calculated based on the concentration and dilution factors, as is well understood by one with ordinary skill in the art. For example, a 250× concentration factor is described in the conventional EPA 537.1 method using WAX columns, and a 1 in 5 dilution factor corresponding to 20% sample input (FIG. 3C) would result in an effective 50× concentration factor, transforming the detection limit of, for example, PFOA, from 1.56 ppm to 31.2 ppb. These limits of detection are assessed as acceptable levels for detection, indicating that the indicator displacement assay will be useful and advantageous as a total PFAS sensor which does not distinguish between different PFAS compounds. It was established that the PFAS compounds can be detected as samples in water or methanol.
Example 4: Determination of Specificity of the Indicator Displacement Assay—This example describes an assessment of the specificity of the assay for detecting PFAS compounds relative to analogous non-fluorinated compounds. Octanoic acid, which is similar in size to PFOA but lacking fluoride substituents, and humic acid, a loose assembly of aromatic polymers of varying acidity and reactivity which are found in environmental samples and formed during long-term decomposition of biomass, were tested in an indicator displacement assay using polymeric beta-cyclodextrin and ANS as an indicator molecule.

There is literature support for there being a greater affinity of monomeric cyclodextrins for PFAS over their analogous non-fluorinated hydrocarbon equivalents of similar size as indicated by equilibrium binding constants (Wilson and Verrall, J. Phys. Chem B., 1997, 101, 9270) and data from competition studies (Xing et al., J. Phys. Chem B., 2007, 111, 8089).

In this experiment, a normalized signal indicator displacement assay was examined with respect to concentration (ppm) of PFOA, octanoic acid, and humic acid using HPLC grade methanol. Stock solutions of 20,000 ppm of PFOA, octanoic acid, and humic acid stock were prepared in 2 mL of HPLC grade methanol in a glass vial. The stock solution was then used to prepare a serial dilution series. After mixing and vortexing, each serial dilution was mixed 10 times with a pipette and 20 μL was pipetted out and added to PCR tubes as described in Example 3. The solution was then mixed using a vortex mixer and incubated at room temperature for 2 hours in a box to prevent photobleaching. Aliquots of 90 μL of each solution were then transferred into a black plate and measured using a spectrophotometer with excitation of 375 nm and emission of 477 nm.

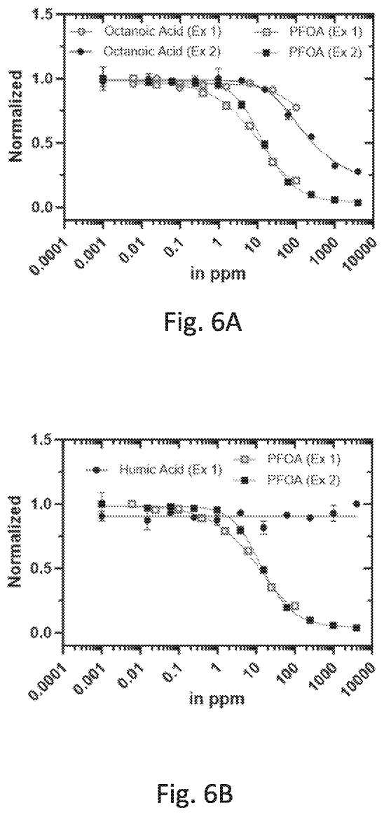

FIG. 6A demonstrates minimal response to octanoic acid below 100 ppm. This concentration level is far above what is typically seen in environmental samples and indicates that octanoic acid, if present, would not be likely to compete with PFAS compounds with respect to binding to the polymeric β-cyclodextrin. This indicates that the indicator displacement assay based on polymeric β-cyclodextrin and ANS is not subject to interference by an analogous non-fluorinated compound. Likewise, the assay does not detect humic acid, which is common in environmental water samples (FIG. 6B). This provides a basis for a prediction that the indicator displacement assay using polymeric β-cyclodextrin and an indicator molecule which binds thereto, will be found to be sufficiently specific to detect common PFAS compounds at low levels in environmental samples.
Example 5: Concentration of Environmental PFAS Samples Prior to Analysis by Indicator Displacement Assays and other Assays—In prior investigations, the inventors have encountered unknown interferents when using non-treated water samples. As such, in order to lower the limit of detection and remove various contaminants to improve detection of PFAS compounds in water samples, a concentration process and system was developed to improve the sample quality prior to analysis by indicator displacement assays or other assays such as ELISAs. This process is configured for field use and represents a significant improvement over the standard EPA concentration protocol (EPA method 1633) which uses solid phase extraction (SPE) cartridges. The pre-concentration process described below is described in context of its use with an indicator displacement assay. However, the skilled person will understand that it may be modified following the re-suspension step for compatibility with PFAS detection assays other than indicator displacement assays, if desired.

The pre-concentration process includes the steps of collecting a water sample into an empty container which is free of PFAS compounds. An aliquot of the sample is applied to a weak anion exchange column (Agilent-Bond Elut PFAS WAX) to bind the PFAS compounds to the column at a controlled speed and the column is dried for about 10 minutes. Next, a syringe (10 mL) is used to add a 10 mL volume of 2% ammonium hydroxide in methanol to the column to elute the PFAS compounds from the column into a collection tube (sodium hydroxide may be used in place of ammonium hydroxide). The collection tube is then dried to complete evaporation of the methanol. The dried PFAS compounds in the collection tube are then re-suspended into 0.1 mL of HPLC-grade methanol and transferred into another reduced volume tube, from which 20 μL aliquots are taken and transferred into reaction tubes containing the reaction mixture. For an indicator displacement assay, the reaction mixture includes a cyclodextrin host molecule and an indicator molecule under conditions where the indicator molecule becomes bound within the cyclodextrin host molecule. In an alternative embodiment, a mixture of the cyclodextrin and the indicator molecule is added directly to the dried collection tube, to generate a sample of PFAS compound(s), which, if present would be more highly concentrated.

A stock solution of 2 mg/ml of AFFF Chemguard 3% solution was prepared in glass vials, using respectively 1 mL of HPLC grade methanol and WAX-column conditioned methanol. Conditioned methanol is used in the EPA 1633 process which uses WAX columns. The “conditioned” methanol is closer to the real “background” that would be observed from a sample instead of what pure methanol would be. The stock solutions were then used to prepare serials dilution into the respective methanol sample. After mixing and vortexing, each serial dilution was mixed 10 times with a pipette and 20 μL was pipetted out and added to PCR tubes as described in Example 3. The solution was then mixed using a vortex mixer and incubated at room temperature for 2 hours in a box to prevent photobleaching. A 90 μL aliquot of each solution was then transferred into a black plate and measured using a spectrophotometer with excitation of 375 nm and emission of 477 nm.

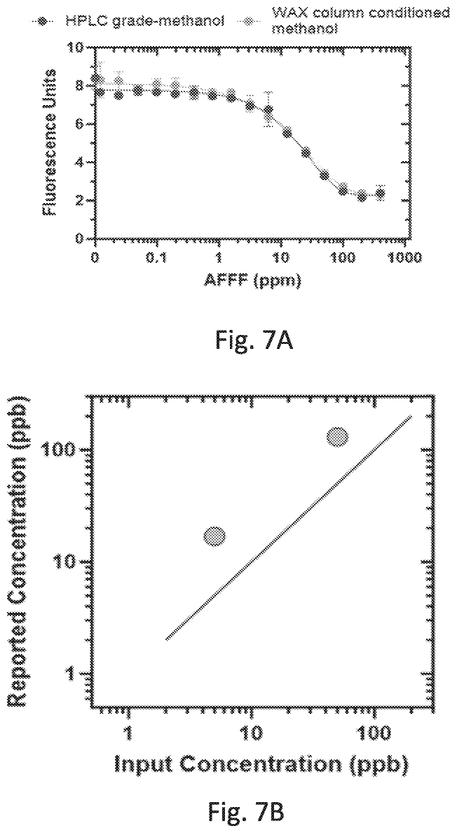

FIG. 7A is a comparison of detection of AFFF using HPLC grade methanol and WAX column conditioned methanol, showing that both solvents provide similar results. This indicates that there is no interference introduced into the system through the use of WAX columns and a method such as described above.

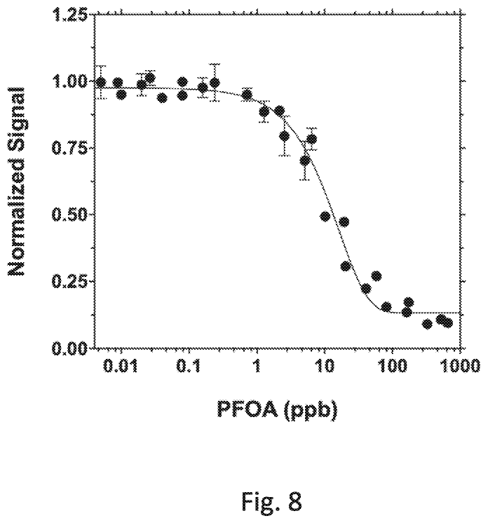

FIG. 7B shows a plot of reported concentration of PFOA using two different input concentrations. The input concentration is calculated by diluting a PFOA stock solution into milliQ™ water. The concentration was extrapolated using the standard curve presented in FIG. 8 to provide a prophetic indicator displacement assay response based on the data of FIG. 7B, normalizing the signal and applying a concentration factor of 3,066× (based on a concentration system that reaches 15,333-fold concentration and the associated 1:5 dilution when the concentrate is added to the indicator displacement assay system. The line shown on the plot of FIG. 7B represents a 1:1 response factor and is provided for illustration purposes only.

It was determined that this process can increase the concentration by 1.53×10⁴, which when combined with standard curves for PFOA, provides a system detection limit of approximately 1 ppb (FIG. 8).

Table 1 provides details of conditions and materials used in the standard EPA 1633 process and in three different process embodiments developed by the present inventors, which were found to be better adapted for conducting the pre-concentration process in the field.

TABLE 1
Conditions and Materials for Pre-Concentration of PFAS Samples

| Method | EPA 1633 | Method 1 | Method 2 | Method 3 |
|---|---|---|---|---|
| Sample | 250 mL | 200 mL at 360 | 2 L at 4 ppb | 2 L at 0.1 ppb |
| Volume | | ppb PFOA in | PFOA in | PFOA in |
| | | MilliQ ™ water | MilliQ ™ water | MilliQ ™ water |
| Columns | Agilent - Bond | Agilent - Bond | Agilent - Bond | Agilent - Bond |
| used | Elut PFAS | Elut PFAS | Elut PFAS | Elut PFAS |
| | WAX, 500 mg, | WAX, 150 mg, | WAX, 500 mg, | WAX, 500 mg, |
| | 6 mL | 6 mL | 6 mL(2 | 6 mL(2 |
| | | | columns) | columns) |
| Conditioning | 10 mL | 10 mL | 10 mL | 10 mL |
| | methanol | methanol | methanol | methanol |
| | followed by 10 | followed by 10 | followed by 10 | followed by 10 |
| | mL of 0.1M | mL of 0.1M | mL of 0.1M | mL of 0.1M |
| | phosphate | phosphate | phosphate | phosphate |
| | buffer, pH 7.0 | buffer, pH 7.0 | buffer, pH 7.0 | buffer, pH 7.0 |
| Rinsing | 10 mL of 1 g/L | 10 mL of 1 g/L | 10 mL of 1 g/L | 10 mL of 1 g/L |
| | ammonium | ammonium | ammonium | ammonium |
| | acetate in | acetate in | acetate in | acetate in |
| | MilliQ ™ water | MilliQ ™ water | MilliQ ™ water | MilliQ ™ water |
| Flow | 5 mL/min | 3.6 mL/min | 3.6 mL/min | 3.6 mL/min |
| Elution | 2% ammonium | HPLC methanol | HPLC methanol | HPLC methanol |
| buffer | hydroxide in | with 2 mM | with 2% NH₄OH | with 2% NH₄OH |
| | MeOH | NaOH | | |
| Elution | 2 × 5 mL | 10 mL | 10 mL per | 10 mL per |
| volume | | | column - total | column - total |
| | | | 20 mL | 20 mL |
| Elution | Vacuum | Manual - | Manual - | Manual - |
| method | pressure 3 to 5 | syringe | syringe | syringe |
| | inHg | | | |
| Drying | Gentle stream | Heat block, | Heat block, | Heat block, |
| method | of nitrogen at | 60°C | 60 °C with air | 60 °C with air |
| | 55 to 60 °C | | pump | pump |
| | until dry | | | |
| Re- | 1 mL | 200 μL | 200 μL | 100 μL |
| suspension | | | | |
| volume | | | | |
| Re- | 80/20 | HPLC Methanol | HPLC Methanol | HPLC Methanol |
| suspension | MeOH/water | | | |
| buffer | | | | |
| Time | 1 h 30 | 10 hours | 6 hours | 6 hours |

Example 6: Investigation of an Electrochemical Indicator Molecules—This example describes an investigation of an indicator molecule which can be detected electrochemically instead of a fluorescent dye indicator molecule. Ferrocene is a stable redox probe which includes a Fe(II) ion coordinated by two cyclopentadiene rings and is known to proceed through many electrochemical cycles, generating oxidized (ferrocenium) and reduced (ferrocene) forms, while remaining intact. This makes ferrocene and its derivatives ideal for electrochemical sensing. Due to ferrocene's hydrophobic nature, a methanolic derivative known as ferrocenemethanol (FcMeOH) was tested. The Applicant has significant experience with the electrochemistry of para-aminophenol (PAP), and it was decided to investigate PAP as an indicator molecule in the indicator displacement assay as well. Cyclic voltammetry was performed on screen printed carbon electrodes in 0.2 M sodium phosphate buffer, pH 7.

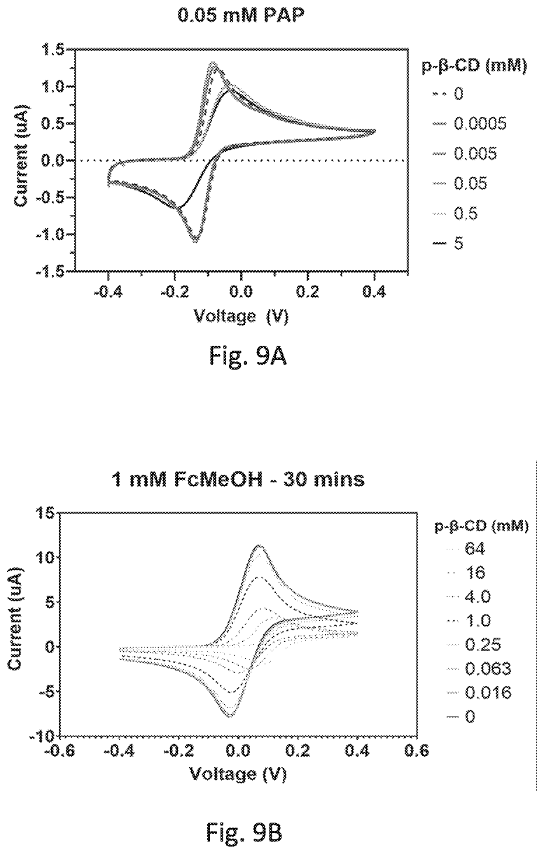

It was established that both FcMeOH and PAP can form guest-host complexes with polymeric-β-cyclodextrin, with their inclusion into the cyclodextrin creating a change in current as demonstrated by cyclic voltammetry in FIGS. 9A (PAP) and 9B (FcMeOH). When the electrochemical indicator molecules are bound to polymeric-β-cyclodextrin, the current is low. The current increases upon displacement and release of the electrochemical indicator molecule from the polymeric-β-cyclodextrin with binding of PFOA to the polymeric-β-cyclodextrin. All electrochemical testing was performed with screen printed electrodes, using a carbon working electrode and counter electrode with a silver/silver chloride reference electrode. As predicted, when the concentration of the cyclodextrin was increased, the current observed from the redox probe (either PAP or FcMeOH) decreased. FcMeOH was chosen for subsequent experiments because it has a more predictable electrochemical response. FcMeOH is more stable than PAP, produces more predictable electrochemical responses, and there are more derivatives available to allow for fine tuning of the cyclodextrin interaction which can be used to fine tune of the limit of detection of the indicator displacement assay.

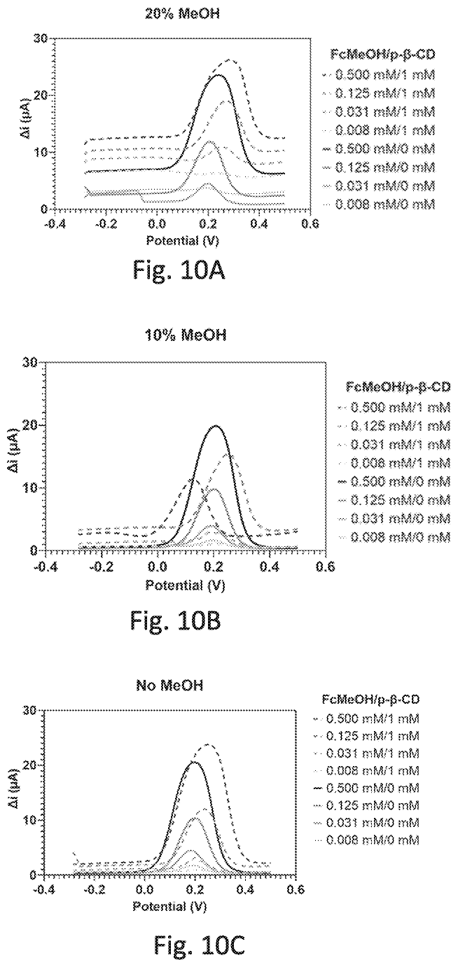

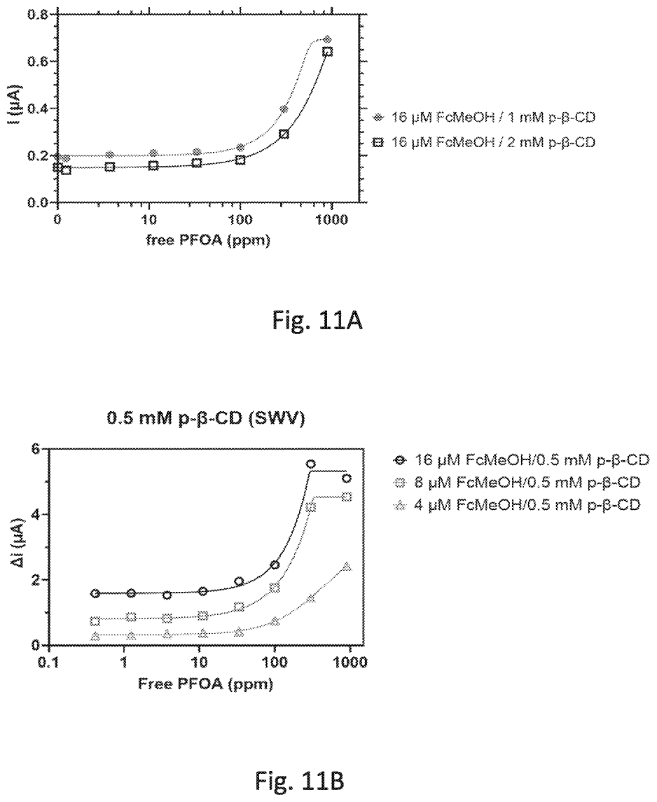

This electrochemical response system was also tested for its response through square wave voltammetry, which is known to one skilled in the art as a more sensitive analytical tool for electrochemical detection relative to the more commonly used cyclic voltammetry method. An example set of square wave voltammetry responses is shown in FIGS. 10A-C, to determine the effect of inclusion of 10% and 20% methanol in a solution of 0.1 M KCl at different concentration ratios of FcMeOH/β-cyclodextrin. While cyclic voltammetry tests demonstrated a concentration dependent increase in signal with PFOA, the sensitivity was initially very poor. To lower the limit of detection, the concentrations of both polymeric-β-cyclodextrin and FcMeOH were required to be lowered. As this causes a decrease in current, it became more important to optimize the system for square wave voltammetry. Both square wave and cyclic voltammetry were used in a preliminary electrochemical indicator displacement assay against PFOA (FIGS. 11A and 11B). The initially determined limits of detection are not significantly lower than what was seen in the fluorescence assays, but this could be a matter of optimizing the assay conditions and/or utilizing a different redox probe such as ferrocenecarboxylic acid, which has a different affinity towards the β-cyclodextrin than FcMeOH.
Example 7: Testing of Environmental Water Samples Suspected of Contamination with PFAS Compounds from Aqueous Film-Forming Foam to Guide Remediation Efforts—This brief prophetic example describes how the indicator displacement assay could be used to rapidly analyze a series of water samples collected from an area around a site which is suspected of being contaminated with PFAS compounds as a result of a major hydrocarbon fire which was previously suppressed using aqueous film forming foam which contains PFAS compounds. Water samples are collected from various water sources within the site and concentrated in the field according to the process described in Example 5. The concentrated samples are subjected to an indicator displacement assay in the field using ANS as a fluorescent indicator molecule. For each of the environmental samples tested, the indicator displacement assay identifies a subset of samples which indicate the general presence and concentration of unspecified PFAS compounds but does not identify the PFAS compounds individually. The results of this broad survey of environmental samples permits the testing team to draw the conclusion that one particular region of the survey site has a water source with a significantly higher concentration of unspecified PFAS compounds than other water sources of the site. The results of this broad survey are provided to a remediation team, enabling the remediation team to focus on that particular water source for remediation, which could be conducted by applying materials which sequester PFAS compounds to the water source. One major advantage of providing a rapid survey of unspecified PFAS compounds is that the process can provide useful results on a timescale which is weeks to months faster than conventional LC-MS, which requires sending samples to laboratories. Furthermore, the molecular components of the assay are relatively inexpensive and the fluorescence and electrochemical detectors are also inexpensive compared to LC-MS equipment. This enables widespread testing of environmental sites to be performed at a relatively low total cost.

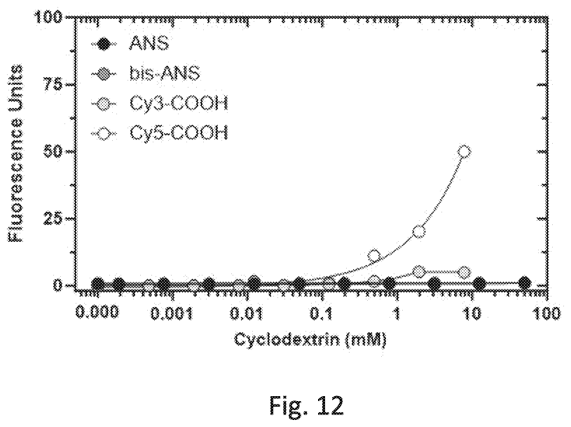

This example demonstrates how a rapid broad survey conducted in the field to identify concentration levels of unspecified PFAS compounds is useful for remediation efforts.
Example 8: Initial Investigation of α-Cyclodextrin as a Scavenger—Identification of Indicator Molecules—The inventors postulated that interferents could bind more effectively to α-cyclodextrin than to β-cyclodextrin and that α-cyclodextrin could therefore be an effective scavenger of interferents that interfere with the indicator displacement assays described herein. It was necessary to investigate if some of the preferred indicator molecules also bind to α-cyclodextrin because such binding would have a detrimental effect on the indicator displacement assay. FIG. 12 shows the results of testing of the binding of different fluorescent molecules to increasing concentration of monomeric α-cyclodextrin (CAS: 10016-20-3). The fluorescence signal of each of the indicator molecules ANS, bis-ANS Cy3-COOH) (a carboxylic acid derivative of Cy3 dye) and Cy5-COOH) (a carboxylic acid derivative of Cy5 dye) is plotted as a function of the concentration (mM) of monomeric α-cyclodextrin. A 10 mM stock solution of each of the fluorescent indicator molecules was prepared in 2 mL of DMSO suitable for HPLC in glass vial. In parallel, a 100 mM monomeric α-cyclodextrin stock was prepared in 10 mL of milliQ™ water in a Falcon™ tube. The stock solution was then used to prepare serial dilution series in milliQ™ water. After mixing and vortexing, each serial dilution was mixed 10 times with a pipette and 50 μL was pipetted out and added to PCR tubes containing 50 μL of the different fluorescent molecule tested (final concentration in milliQ™: 0.008 mM ANS, 0.008 mM Cy3-COOH), 0.004 mM bis-ANS and 0.004 mM Cy5-COOH)). The solution was then mixed using a vortex mixer and incubated at room temperature for 2 hours in a box to prevent photobleaching. Then 90 μL of each solution was transferred into a black plate for fluorescence measurements using a spectrophotometer.

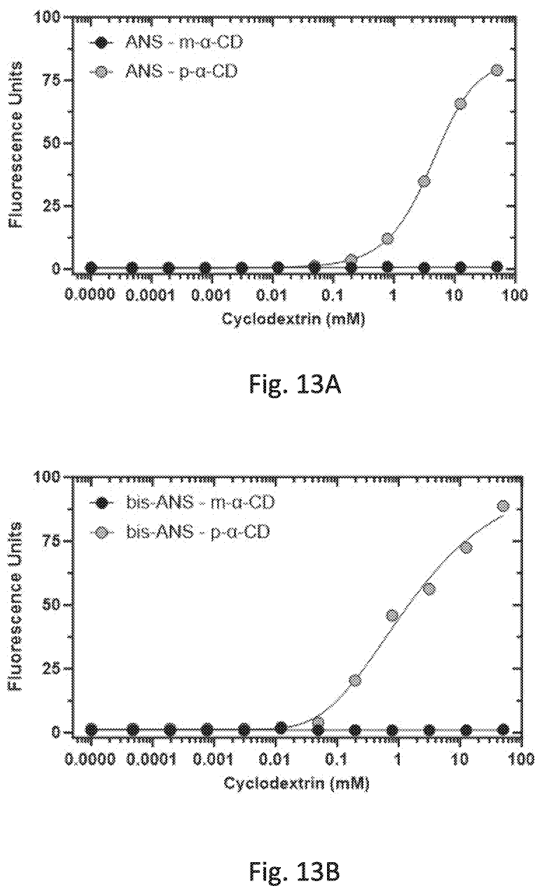

The results indicate that significant binding of Cy5-COOH) to monomeric α-cyclodextrin occurs and some relatively minor binding of Cy3-COOH) to monomeric α-cyclodextrin occurs. No appreciable binding of ANS and bis-ANS to monomeric α-cyclodextrin occurs. As a result, ANS and bis-ANS are used in subsequent experiments described below. Cy3-COOH) and Cy5-COOH) were not investigated further as indicator molecules for indicator displacement assays that include a scavenger.
Example 9: Comparison of Monomeric and Polymeric α-Cyclodextrin as Potential Scavengers of Interferents—Testing of the binding of ANS and bis-ANS to monomeric and polymeric versions of α-cyclodextrin were investigated to determine which of these compounds is best suited for use as a scavenger of interferents. The result of testing of the binding of ANS is shown in FIG. 13A and the result of testing of binding of bis-ANS is shown in FIG. 13B with the response to increasing concentration of the monomer alpha cyclodextrin (CAS: 10016-20-3) and the polymer alpha cyclodextrin (CAS: CY1009-ACDPS) being indicated. The fluorescence signal of each molecule as a function of the concentration (mM) of monomeric alpha cyclodextrin (black circle) and polymeric alpha cyclodextrin (gray circle) are shown. A 10 mM stock of ANS and bis-ANS was prepared in 2 mL of DMSO suitable for HPLC in glass vial. In parallel, a 100 mM monomeric alpha cyclodextrin stock (monomeric or polymeric) was prepared in 10 ml of milliQ™ water in a Falcon™ tube. The stock solution was then used to prepare serial dilution series in milliQ™ water. After mixing and vortexing, each serial dilution was mixed 10 times with a pipette and 50 μL was pipetted out and added to PCR tubes containing 50 μL of the different fluorescent molecule tested (final concentrations in milliQ™: 0.008 mM ANS, and 0.004 mM bis-ANS). The solution was then mixed using a vortex mixer and incubated at room temperature for 2 hours in a box to prevent photobleaching. Then 90 μL of each solution was transferred into a black plate, and measured using a spectrophotometer.

The results shown in FIGS. 13A and 13B indicate that significant binding of ANS and bis-ANS to polymeric α-cyclodextrin occurs while significant binding of ANS and bis-ANS to monomeric α-cyclodextrin does not occur. This result indicates that monomeric α-cyclodextrin is better suited for use as a scavenger in an indicator displacement assay than polymeric α-cyclodextrin.
Example 10: Investigation of Monomeric α-Cyclodextrin as an Interferent Scavenger in an Indicator Displacement Assay—In this series of experiments, indicator displacement assays were conducted to determine if interferents reduce the sensitivity of the indicator displacement assay by reducing the fluorescence signal and if reduced sensitivity can be reversed or rescued by inclusion of monomeric α-cyclodextrin as an interferent scavenger.

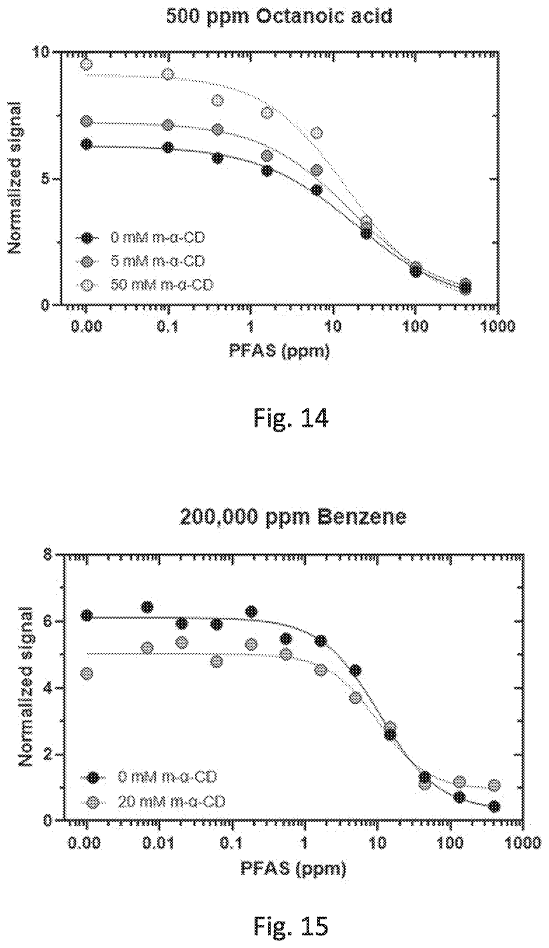

FIG. 14 is a plot of fluorescence signal as a function of increasing concentration (ppm) of PFAS (PFOA) in the presence of 500 ppm octanoic acid. FIG. 15 is a plot of fluorescence signal as a function of increasing concentration (ppm) of PFAS (PFOA) in the presence of 200,000 ppm of benzene (right). A 10 mM stock of ANS was prepared in 2 mL of DMSO suitable for HPLC in glass vial. In parallel, a 100 mM monomeric α-cyclodextrin stock solution was prepared in 10 mL of milliQ™ water in a Falcon™ tube. The stock solution of PFOA (2 mg/mL in HPLC grade methanol) was used to prepare serial dilution series in milliQ™ water. After mixing and vortexing, each serial dilution was mixed 10 times with a pipette and 50 μL was pipetted out and added to PCR tubes containing 50 μL of mastermix (final concentration in milliQ™: 0.008 mM ANS, 0.25 mM polymeric β-cyclodextrin and corresponding concentration of monomeric α-cyclodextrin) containing the tested interferent (octanoic acid at 1,000 ppm for a final concentration of 500 ppm or benzene at 400,000 ppm for a final concentration of 200,000 ppm). The solution was then mixed using a vortex mixer and incubated at room temperature for 2 hours in a box to prevent photobleaching. Then 90 μL of each solution was transferred into a black plate, and fluorescence was measured using a spectrophotometer (excitation of 375 nm and emission of 477 nm).

The results shown in FIG. 14 indicate that some binding of octanoic acid to polymeric β-cyclodextrin causes reduction in the normalized fluorescence observed in the absence of PFAS and monomeric α-cyclodextrin, where the fluorescence reading of about 6 is reduced relative to the normalized fluorescence reading near 10, when the assay mixture includes 50 mM monomeric α-cyclodextrin. An intermediate normalized fluorescence reading of about 7.5 is observed when an intermediate concentration of 5 mM monomeric α-cyclodextrin is included in the assay mixture. A drop in fluorescence signal is observed in each case with increasing concentrations of PFAS (PFOA). These results indicate that the model interferent octanoic acid interferes with the indicator displacement assay by binding to polymeric β-cyclodextrin and that this effect can be reversed by inclusion of monomeric α-cyclodextrin as an interferent scavenger.

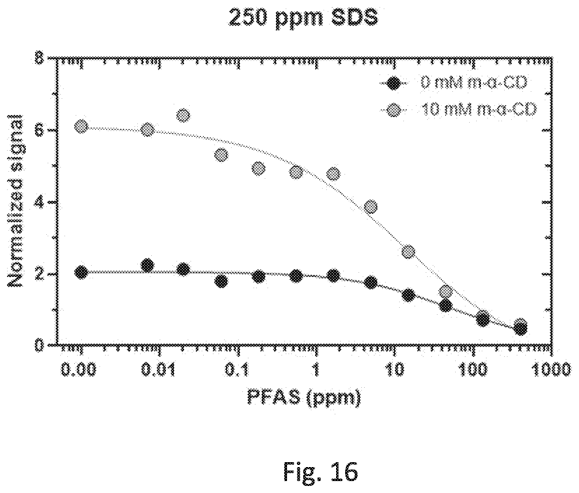

The results shown in FIG. 15 indicate that in the presence of benzene as a model interferent, inclusion of monomeric α-cyclodextrin as an interferent scavenger does not significantly improve the sensitivity of the assay. This is not surprising because it was postulated that benzene is too large to bind within the cavity of polymeric β-cyclodextrin.
Example 11: Investigation of Monomeric α-Cyclodextrin as an Interferent Scavenger in an Indicator Displacement Assay with SDS as a Model Interferent—In this experiment, an indicator displacement assay was conducted in the presence of the surfactant sodium dodecyl sulfate (SDS) as a model interferent. As noted hereinabove, surfactants with alkyl chains of at least 6 carbons are common interferents at environmental sites contaminated with PFAS compounds and have been postulated by the inventors to have a high likelihood of interfering with the sensitivity of embodiments of the indicator displacement assay described herein. In FIG. 16, the results of an indicator displacement assay conducted in the presence of 250 ppm SDS with and without monomeric α-cyclodextrin are shown in a plot of normalized fluorescence signal vs. concentration of PFAS (PFOA). A 10 mM stock of ANS was prepared in 2 mL of DMSO suitable for HPLC in glass vial. In parallel, a 100 mM monomeric α-cyclodextrin stock was prepared in 10 mL of milliQ™ water in a Falcon™ tube. The stock solution of PFOA (2 mg/mL in HPLC grade methanol) was used to prepare serial dilution series in milliQ™ water. After mixing and vortexing, each serial dilution was mixed 10 times with a pipette and 50 μL was pipetted out and added to PCR tubes containing 50 μL of mastermix (final concentration in milliQ™: 0.008 mM ANS, 0.25 mM polymeric β-cyclodextrin and corresponding concentration of monomeric α-cyclodextrin) containing 500 ppm SDS for a final concentration of 250 ppm). The solution was then mixed using a vortex mixer and incubated at room temperature for 2 hours in a box to prevent photobleaching. Then 90 μL of each solution was transferred into a black plate and measured using a spectrophotometer (excitation of 375 nm and emission of 477 nm).

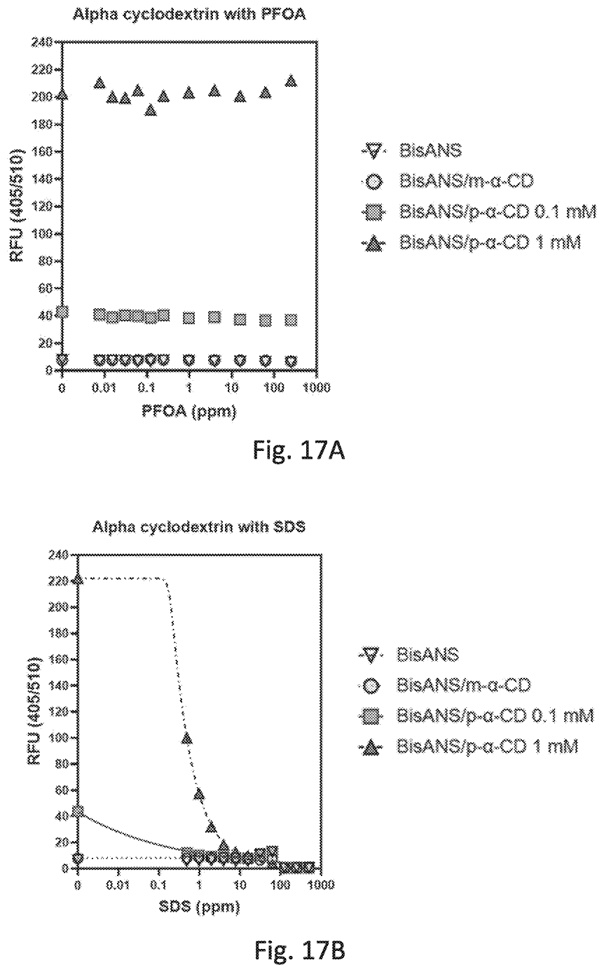

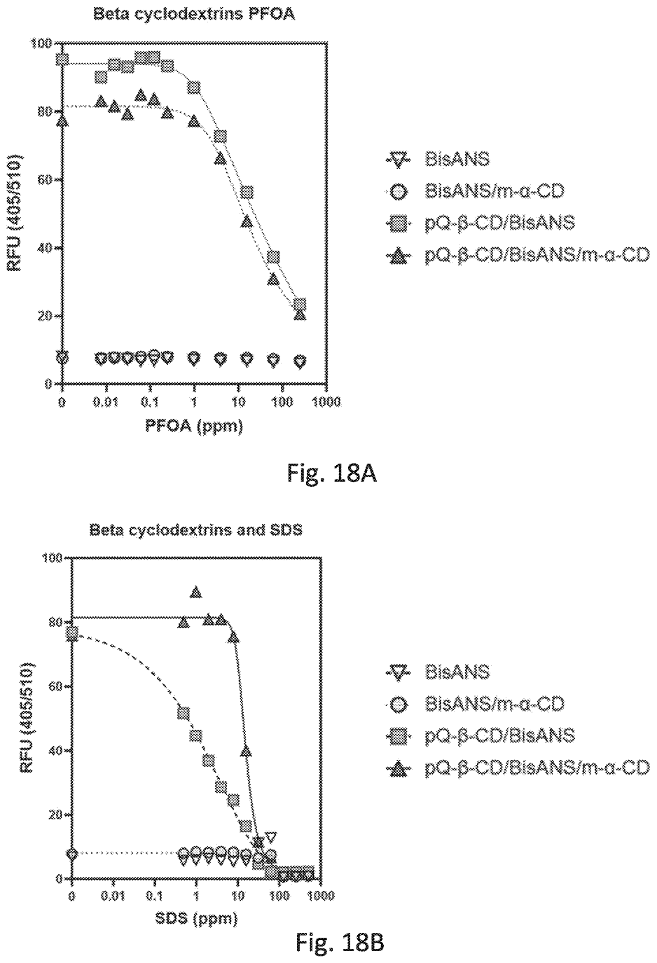

The results indicate that in the absence of the monomeric α-cyclodextrin scavenger, the normalized signal is at about 2 and is only lowered slightly at the highest concentrations of PFAS. This demonstrates that SDS competes with ANS for binding to the polymeric β-cyclodextrin, which serves as the basis for the indicator displacement assay. On the other hand, in the presence of 10 mM monomeric α-cyclodextrin, the normalized signal in the absence of PFAS is at about 6 and decreases consistently with increasing concentrations of PFAS. These results indicate that the monomeric α-cyclodextrin scavenger plays an important role in rescuing the utility of the indicator displacement assay when the model interferent SDS is included in the assay mixture, with the interferent SDS being bound and sequestered by the scavenger to reduce interference with the assay. It is reasonably predicted by the inventors that similar rescue of the assay will occur with other surfactants and hydrocarbons having alkyl chains of at least 6 carbons.
Example 12: Investigation of the Ability of Monomeric and Polymeric α-Cyclodextrin to Bind to PFOA and Model Interferent SDS—It was deemed necessary to investigate whether monomeric and polymeric versions of α-cyclodextrin are capable of binding to PFOA and to SDS. FIGS. 17A and 17B are plots of relative fluorescence units vs. concentration of PFOA (in FIG. 17A) and concentration of SDS (FIG. 17B). Mastermixes were made from the following stock concentrations: 10 mM of bis-ANS was prepared in DMSO suitable for HPLC in glass vial; 100 mM monomeric alpha cyclodextrin stock was prepared in milliQ™ water in a polypropylene tube; 25 mM polymeric α-cyclodextrin was prepared in milliQ™ water. A stock solution of SDS (0.2 mg/mL in 50% HPLC grade methanol/water) was used to prepare serial dilution series in 50% HPLC grade methanol/water. A stock solution of PFOA (0.5 mg/ml in 50% HPLC grade methanol/water) was used to prepare serial dilution series in 50% HPLC grade methanol/water. After mixing and vortexing, each serial dilution was pipetted into PCR tubes to a volume of 50 μL. Mastermix was added to the SDS and PFOA series at a volume of 50 μL (final concentration in milliQ™: 0.012 mM bis-ANS, 10 mM monomeric alpha-cyclodextrin and 0.1 or 1 mM polymeric α-cyclodextrin). The solution was then mixed using a vortex mixer and incubated at room temperature for 2 hours in a box to prevent photobleaching. Then 50 μL of each solution was transferred into a PCR plate, and measured using a spectrophotometer (excitation of 405 and emission of 510 nm). The results indicate that no appreciable binding of PFOA to the monomeric and polymeric versions of α-cyclodextrin occurs (FIG. 17A). This is a useful result which indicates that these scavenger compounds will not affect the basis for the indicator displacement assay. On the other hand, FIG. 17B shows that SDS significantly binds to polymeric α-cyclodextrin and displaces the indicator molecule bis-ANS at relatively low concentrations of SDS.
Example 13: Investigation of the Ability of Quaternary Amine-Modified β-Cyclodextrin to Bind PFOA and SDS in the Presence and Absence of monomeric α-Cyclodextrin—It was deemed necessary to compare binding of monomeric and polymeric versions of β-cyclodextrin to determine if they are capable of binding to PFOA and to SDS in the presence or the absence of the monomeric α-cyclodextrin scavenger. Fluorescence signal as relative fluorescence units is plotted as a function of increasing concentration (ppm) of PFOA (FIG. 18A) or SDS (FIG. 18B). Mastermixes were made from the following stock concentrations: 10 mM of bis-ANS was prepared in DMSO suitable for HPLC in a glass vial; 100 mM monomeric α-cyclodextrin stock was prepared in milliQ™ water in a polypropylene tube; 5 mM quaternary amine modified polymeric β-cyclodextrin with an average of 1.5 quaternary amine groups per cyclodextrin molecule (hereinafter referred to as “degree of substitution of 1.5 or DS1.5”) was prepared in milliQ™ water. A stock solution of SDS (0.2 mg/mL in 50% HPLC grade methanol/water) was used to prepare serial dilution series in 50% HPLC grade methanol/water. A stock solution of PFOA (0.5 mg/ml in 50% HPLC grade methanol/water) was used to prepare a serial dilution series in 50% HPLC grade methanol/water. After mixing and vortexing, each serial dilution was pipetted into PCR tubes to a volume of 50 μL. Mastermix was added to the SDS and PFOA series at a volume of 50 μL (final concentration in milliQ™: 0.012 mM bis-ANS, 10 mM monomeric α-cyclodextrin and 0.1 or 1 mM polymeric β-cyclodextrin). The solution was then mixed using a vortex mixer and incubated at room temperature for 2 hours in a box to prevent photobleaching. Then 50 μL of each solution was transferred into a PCR plate and measured using a spectrophotometer (excitation of 405 and emission of 510 nm).

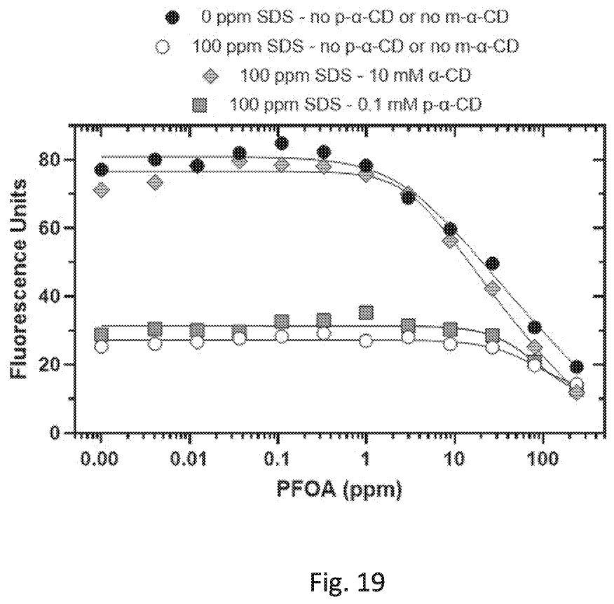

The results shown in FIG. 18A indicate that PFOA does not bind to monomeric α-cyclodextrin but does bind to polymeric quaternary amine modified β-cyclodextrin both in the absence and presence of monomeric α-cyclodextrin. In the initial fluorescence readings, at lower concentrations of PFOA, the combination of quaternary amine modified β-cyclodextrin and monomeric α-cyclodextrin has a slightly lower fluorescence than the mixture without monomeric α-cyclodextrin but the response to higher concentrations of PFOA is similar. The results shown in FIG. 18B indicate that the combination of polymeric quaternary amine modified β-cyclodextrin and monomeric α-cyclodextrin retains fluorescence in the presence of increasing concentrations of SDS until a concentration of about 10 ppm SDS is reached and then the fluorescence decreases. This appears to be a result of SDS overwhelming the scavenging ability of monomeric α-cyclodextrin for the concentration of α-cyclodextrin used in this example. In contrast, polymeric quaternary amine modified-cyclodextrin without monomeric α-cyclodextrin responds to lower concentrations of SDS. This experiment indicates that the use of monomeric α-cyclodextrin as an interferent scavenger in combination with polymeric quaternary amine modified β-cyclodextrin will be effective in improving the sensitivity of the indicator displacement assay in situations where interferent surfactants and hydrocarbons with chains of at least 6 carbons are present.
Example 14: Investigation of the Role of Increasing Concentration of Monomeric and Polymeric α-Cyclodextrin as a Scavenger in an Indicator Displacement Assay—FIG. 19 shows the results of an indicator displacement assay in the presence and absence of 100 ppm SDS with varying amounts of monomeric or polymeric α-cyclodextrin with fluorescence units plotted against concentration of PFOA. A 10 mM stock of bis-ANS was prepared in 2 mL of DMSO suitable for HPLC in glass vial. In parallel, 100 mM monomeric α-cyclodextrin and 100 mM polymeric α-cyclodextrin stock solutions were prepared in 10 mL of milliQ™ water in a Falcon™ tube. The stock solution of PFOA (2 mg/mL in HPLC grade methanol) was used to prepare serial dilution series in milliQ™ water. After mixing and vortexing, each serial dilution was mixed 10 times with a pipette and 50 μL was pipetted out and added to PCR tubes containing 50 μL of mastermix (final concentration in milliQ™: 0.012 mM bis-ANS, 0.015 mM PQ polymeric β-cyclodextrin and corresponding concentration of α-cyclodextrin) containing milliQ™ water or 200 ppm SDS (final concentration of 100 ppm). The solution was then mixed using a vortex mixer and incubated at room temperature for 2 hours in a box to prevent photobleaching. Then 90 μL of each solution was transferred into a black plate, and measured using a spectrophotometer (excitation of 405 and emission of 510 nm).

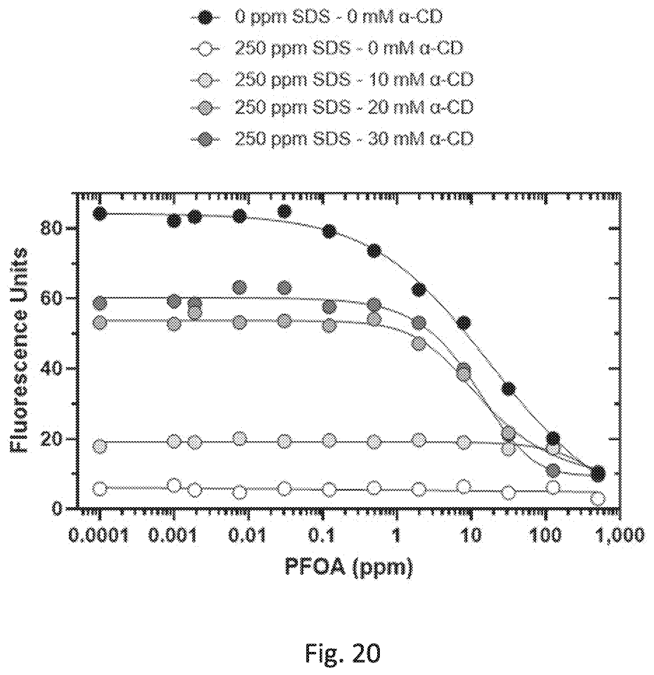

The results indicate that the presence of the model interferent SDS significantly reduces the initial fluorescence signal, demonstrating that SDS is binding to polymeric β-cyclodextrin and interfering with the indicator displacement assay. Inclusion of 10 mM monomeric α-cyclodextrin in the assay reverses this effect. Inclusion of 0.1 mM polymeric α-cyclodextrin fails to reduce this effect. These results indicate that monomeric α-cyclodextrin is an effective scavenger for the model interferent, provided that a sufficient concentration is provided. The results also indicate that polymeric α-cyclodextrin is not an effective scavenger for the model interferent.
Example 15: Investigation of the Role of Increasing Concentration of Monomeric α-Cyclodextrin on the Limit of Detection of an Indicator Displacement Assay—FIG. 20 shows a plot of fluorescence units vs. concentration of PFOA for assays in the absence of SDS, and with 250 ppm of SDS and inclusion of varying concentrations of monomeric α-cyclodextrin. A clear trend is seen where the presence of SDS in the absence of α-cyclodextrin significantly reduces the fluorescence signal and that this effect is increasingly reversed with inclusion of increasing concentrations of monomeric α-cyclodextrin.

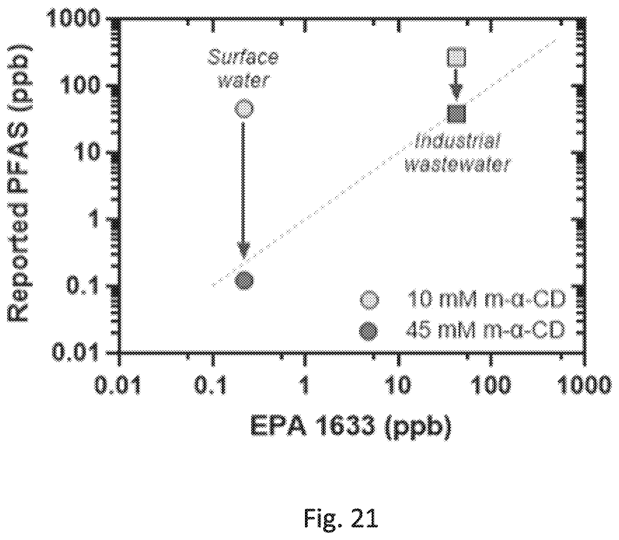

These results confirm that monomeric α-cyclodextrin is an effective scavenger for the model interferent.
Example 16: Investigation of the Role of Increasing Concentration of α-Cyclodextrin on Accuracy of the Indicator Displacement Assay in Analysis of Environmental Samples—FIG. 21 is a plot of reported PFAS concentration in ppb determined using an embodiment of the indicator displacement assay vs. reported PFAS concentration determined using the conventional LC-MS/MS EPA 1633 process for environmental samples. The environmental samples were concentrated via solid phase extraction prior to analysis through the indicator displacement assay. For the indicator displacement assay, stock solutions for the mastermix were prepared in the following manner: 10 mM stock of bis-ANS was prepared in DMSO suitable for HPLC in glass vial; 100 mM monomeric α-cyclodextrin stock was prepared in milliQ™ water; 5 mM of DS1.5 quaternary amine-modified polymeric β-cyclodextrin was prepared in milliQ™ water. These stocks were combined to the following final concentrations in milliQ™: 0.012 mM bis-ANS, 0.15 mM quaternary amine-modified polymeric β-cyclodextrin and corresponding concentration of monomeric α-cyclodextrin (10 or 45 mM). After mixing and vortexing, each serial dilution was mixed 2 times with a pipette and 50 μL was pipetted to PCR tubes containing 50 μL of mastermix. The solution was then mixed using a vortex mixer and incubated at room temperature for 2 hours in a box to prevent photobleaching. Then 50 μL of each solution was transferred into a skirted plate, and measured using a spectrophotometer (excitation of 405 and emission of 510 nm).

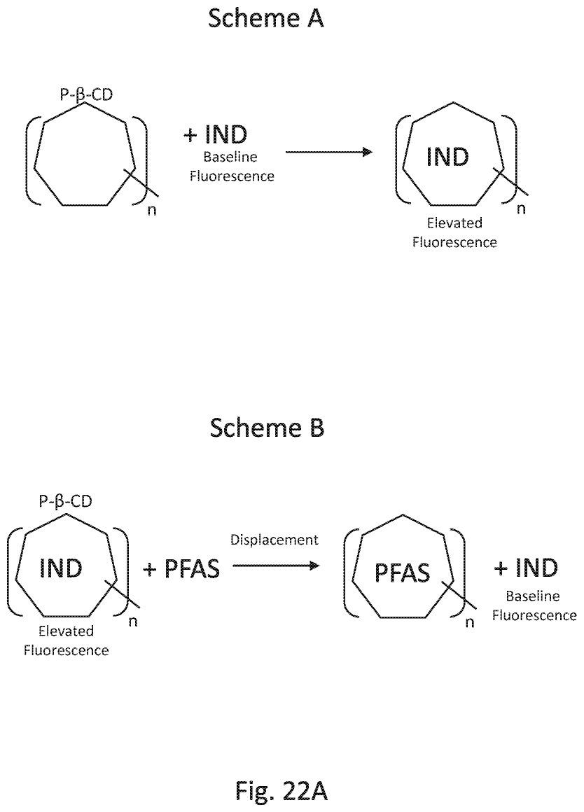

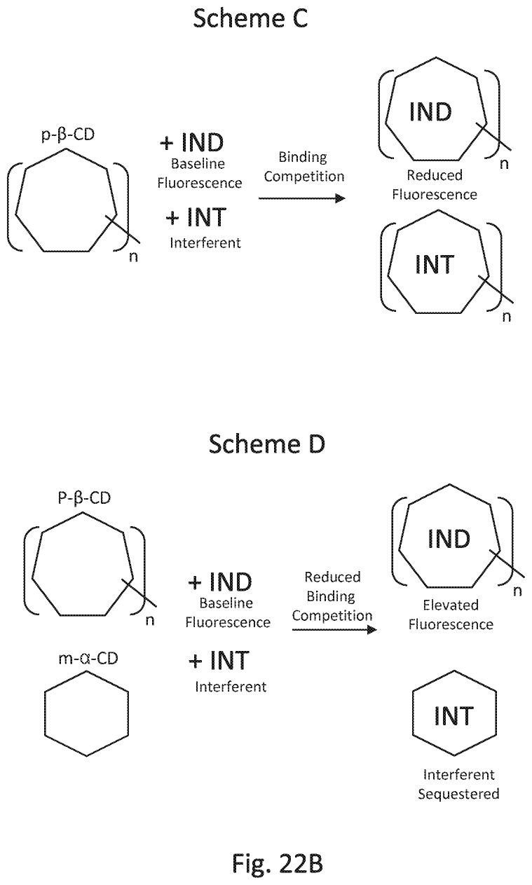

The results indicate that increasing the amount of monomeric α-cyclodextrin brings the concentration determined by the fluorescence signal in one embodiment of the indicator displacement assay in line with the reported concentration determined using the conventional EPA 1633 method which uses LC-MS/MS. This is likely due to multiple non-PFAS organic interferents being present in the samples that overwhelmed the 10 mM concentration of the scavenger monomeric α-cyclodextrin resulting in additional bis-ANS being displaced by the interferents and resulting in artificially high concentrations of PFAS being reported. Increasing the concentration to 45 mM resolved this issue. The concentration of 45 mM monomeric α-cyclodextrin is approaching the solubility limit of monomeric α-cyclodextrin, which is why this concentration was chosen.
Example 17—Overview of Binding Schemes for PFAS, Indicator Molecule and Interferents in the Indicator Displacement Assay—FIGS. 22A and 22B illustrate the binding principles involved in the indicator displacement assay with and without a scavenger exemplified by monomeric α-cyclodextrin (m-α-CD).

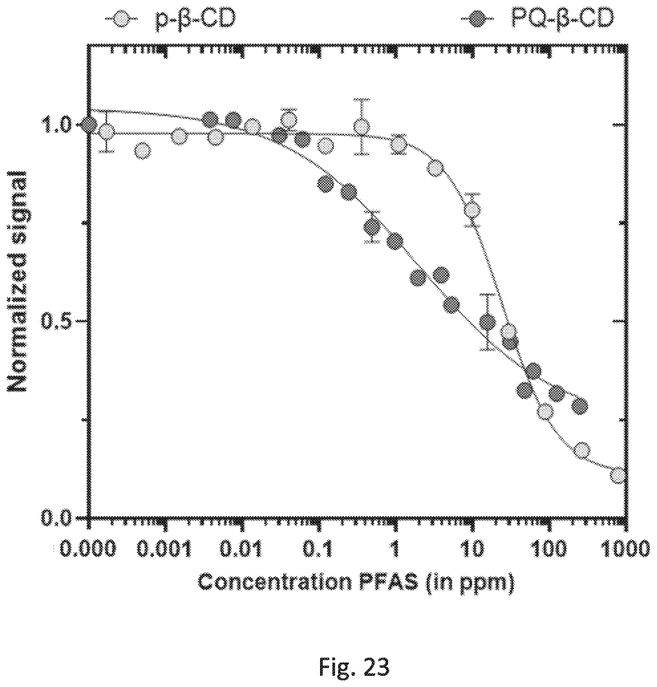

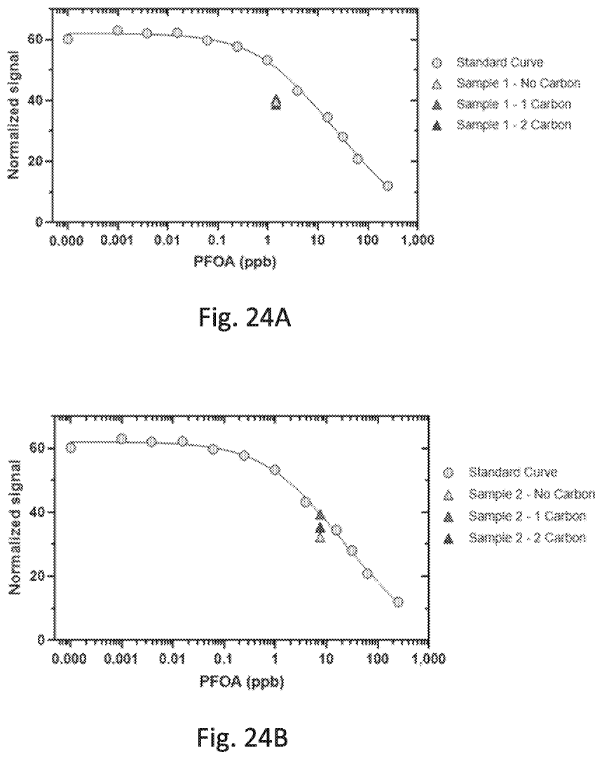

Scheme A of FIG. 22A shows how contact of polymeric β-cyclodextrin (p-β-CD) with an indicator molecule (IND), results in the baseline fluorescence of the indicator molecule becoming elevated as a result of binding of the indicator to the polymeric β-cyclodextrin. Scheme B of FIG. 22A indicates how a PFAS molecule will displace the indicator molecule from the polymeric β-cyclodextrin to result in the fluorescence of the indicator molecule returning to its baseline. Scheme C of FIG. 22B indicates an interference situation where an interferent (INT), which could be a long chain hydrocarbon or a surfactant having a hydrocarbon chain of at least 6 carbons, competes with the indicator molecule for binding to the polymeric β-cyclodextrin. The result is that the fluorescence provided by the complex of the indicator molecule with polymeric β-cyclodextrin is undesirably reduced because a fraction of the available polymeric β-cyclodextrin is occupied by the interferent. Scheme D of FIG. 22B indicates how the undesirable effect of the interferent is mitigated by inclusion of a scavenger molecule exemplified by monomeric α-cyclodextrin. The scavenger molecule sequesters the interferent (INT) such that it cannot compete with the indicator molecule and the result is that the desirable elevated fluorescence resulting from binding of the indicator molecule to polymeric β-cyclodextrin is provided such that the presence of PFAS in a sample being measured using the multiple displacement assay will displace the indicator molecule and reduce fluorescence in a manner which accurately reflects the amount of PFAS in the sample.
Example 18—Comparison of Polymeric β-Cyclodextrin with Polymeric Quaternary Amine-Modified β-Cyclodextrin in an Indicator Displacement Assay—FIG. 23 shows the results of a comparison between polymeric β-cyclodextrin with polymeric quaternary amine-modified β-cyclodextrin in an indicator displacement assay under conditions similar to those of the examples above. The results demonstrate improvement in sensitivity using the DS1.5 quaternary amine modified β-cyclodextrin when compared to unmodified polymeric β-cyclodextrin. Both polymers are approximately 120 kDa in length and include epichlorohydrin linkers. The degree of substitution is an average of 1.5 quaternary amines per cyclodextrin unit. In related experiments (not shown), it has been found that different polymer lengths do not have a significant effect on sensitivity.
Example 19—Blind Testing of PFAS Samples by EPA 1633 and an Indicator Displacement Assay—A collaborator provided a series of six PFAS samples having different concentrations of PFAS molecules at various concentrations. The samples were analyzed by an embodiment of the indicator displacement assay which uses polymeric DS1.5 quaternary amine-modified β-cyclodextrin in triplicate and by the EPA 1633 process which uses LC-MS/MS. The ppt level reported is the sum of all PFAS species detected via the 1633 method (which reports on multiple discrete PFAS species individually).

The results of determination of PFAS concentrations are shown in Table 2 below.

TABLE 2
Blind Sample Testing

| | | | Difference (%) |
|---|---|---|---|
| | | | EPA 1633 vs. |
| | | Indicator | Indicator |
| | EPA 1633 | Displacement | Displacement |
| Sample | (ppt) | Assay (ppt) | Assay |
| Sample A | 15 | N.D. | N/A |
| Sample B | 73 | N.D. | N/A |
| Sample C | 77 | 87 ± 3 | 14 |
| Sample D | 250 | 246 ± 88 | 2 |
| Sample E | 720 | 532 ± 99 | 26 |
| Sample F | 840 | 1001 ± 34 | 19 |
| | | Average | 15 |
| | | Difference: | |
| | | R squared | 0.87 |

The results shown in Table 2 indicate that at least one embodiment of the indicator displacement assay is capable of providing PFAS detection sensitivity at generally the same orders of magnitude as the standard EPA 1633 method which is more time consuming and expensive than projected costs of operating a field deployable indicator displacement assay. This indicates that embodiments of the PFAS indicator displacement assays described herein will be useful for incorporation into field deployable instruments and kits for detection and quantification of PFAS compounds.
Example 20—Pre-Treatment of Environmental Samples with a Carbon Column—To test the effect of pre-treatment of environmental samples with a carbon column, two different samples were centrifuged (10 °C, 20 mins, 4100× rcf) and filtered using a 0.45 μm Nylon syringe filter to remove any debris that could clog the columns. SDVB pre-packed columns (500 mg, 6 mL, Agilent Technologies, cat number: 12255021) were pre-conditioned using 15 mL 100% methanol and 20 mL of milliQ™ water. 100 mL of each sample was loaded onto the column, and washed with 50 mL of milliQ™ and dried for 5 min at room temperature to remove any remaining buffer. PFAS bound to the column was then eluted using 4 mL 100% methanol solution (v/v). The eluate was then transferred onto a pre-packaged carbon column. The resulting eluate was dried and the eluted PFAS compound was resuspended into 50% methanol (v/v) solution. Then 50 μL of sample was added to a black 96 well plate containing 50 μL of assay mastermix (0.012 mM bis-ANS, 0.015 mM polymeric DS1.5 quaternary amine-modified β-cyclodextrin (pQ-β-CD) and 25 mM of α-cyclodextrin). The plate was covered and incubated at room temperature for 1 h. The fluorescence was measured using a spectrophotometer with excitation of 405 nm and emission of 510 nm. Three aliquots of each of the two samples were analyzed using an indicator displacement assay where one control aliquot was not subjected to carbon pre-treatment, one aliquot was subjected to one carbon column treatment and one aliquot was subjected to two column treatments. The results shown for the first sample in FIG. 24A indicates that the carbon column pre-treatment step had essentially no effect on the detection of PFAS by the indicator displacement assay. On the other hand, the results shown for the second sample in FIG. 24B indicates that the carbon column pre-treatment step improved the condition of the sample such that the detected PFAS concentration became aligned with the standard curve. The results of this experiment indicate that a carbon column pre-treatment step may be advantageous in some cases and at least does not appear to generate a detrimental effect on the assay.
Example 21—Investigation of the Effect of Washing of a Hydrophobic Column Prior to Elution of Bound PFAS on the Indicator Displacement Assay—As another potential pre-treatment step to remove uncharacterized interferents during hydrophobic column chromatography, three different washing arrangements for washing bound PFAS sample from an SDVB column were investigated; water, 5% methanol in water and a 1:1 solution of 5% methanol and 0.1 M formic acid. An SDVB 96-well solid phase extraction plate (25 mg, 2 mL, Agilent Technologies, cat number: A3961025) was conditioned with 1 mL of methanol and equilibrated with 1 mL of MilliQ™ water. Then 10 mL of milliQ™ non-spiked (0 ppm PFAS expected) and spiked with 10 ppm PFOA were passed through the wells of the 96-well plate. For the water wash, 3 mL of milliQ™ water was passed (black circle); for the 5% methanol wash, the SVDB resin was washed with 1 mL 5% methanol (v/v) followed by 2 mL of milliQ™ water (dark gray circle) and finally for the methanol and formic acid wash, the resin was first washed with 1 mL 5% methanol solution (v/v), followed by 1 mL of 0.1 M formic acid/methanol (1:1) and 1 mL of milliQ™ water (light gray circle). PFAS was eluted from the resin using 0.4 μL of 100% methanol. Eluted samples were dried out at 90 °C and reconditioned in 100 μL of 50% methanol (v/v). 25 μL of sample was then added to a black 96-well plate containing 25 μL of mastermix: 0.012 mM bis-ANS, 0.015 mM polymeric DS1.5 quaternary amine-modified β-cyclodextrin (pQ-β-CD) and 25 mM of α-cyclodextrin). The plate was covered and incubated at room temperature for 1 h. The fluorescence was measured using a spectrophotometer (excitation of 405 nm and emission of 510 nm).

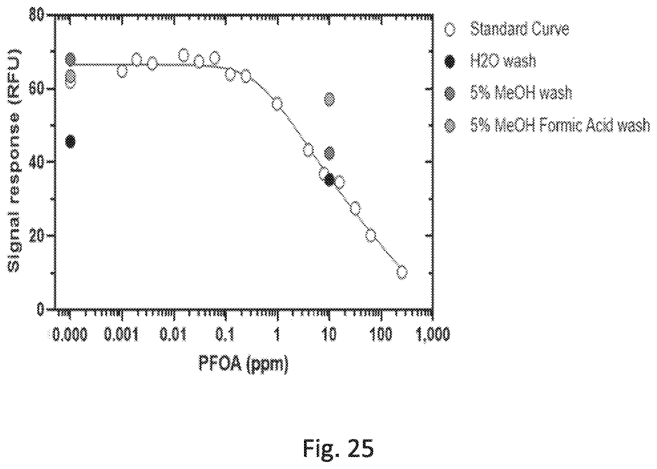

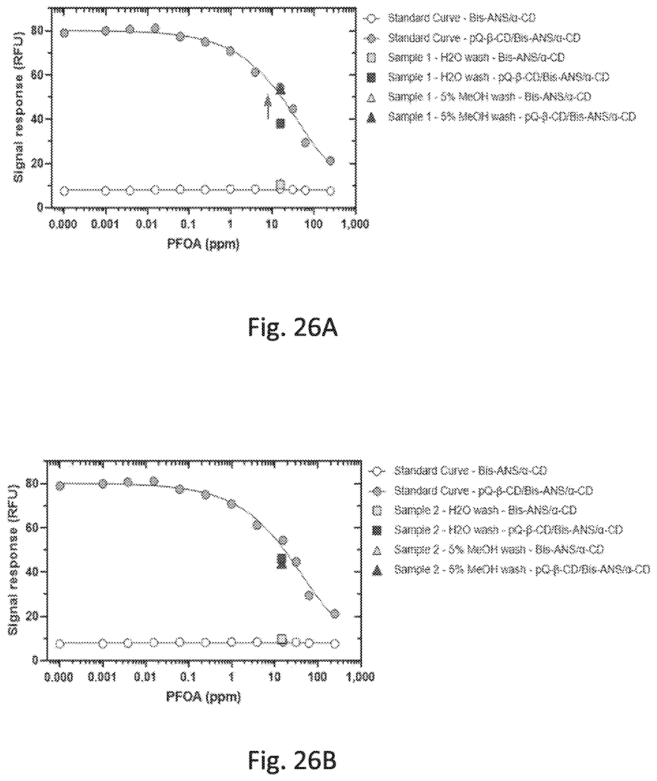

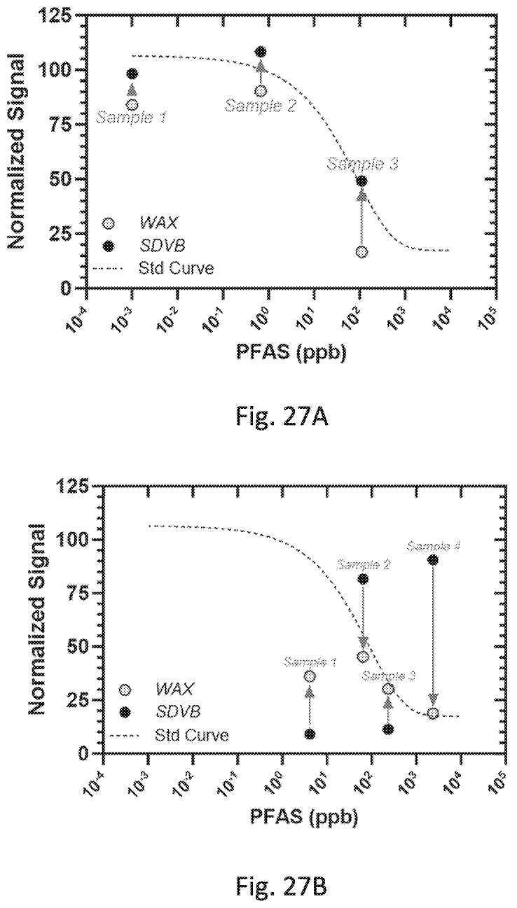

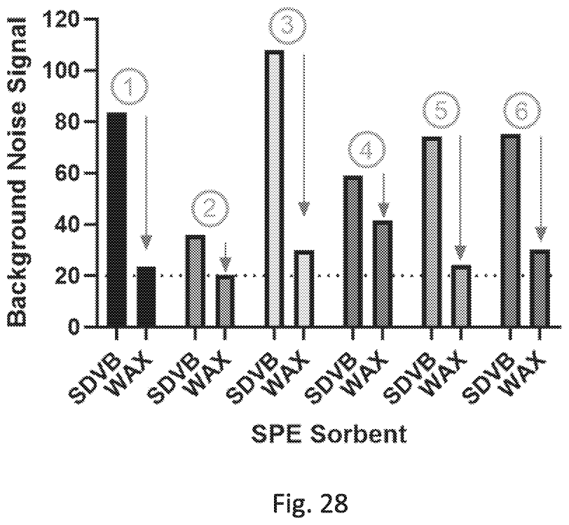

The results of this experiment are shown in FIG. 25, where it is seen that the 5% methanol wash provided the effect of improving the condition of the sample such that it aligns the detected PFAS concentration with the standard curve. On the other hand, washing with water and washing with the 5% methanol and formic acid provide sub-optimal results, where the detected concentrations do not align with the standard curve. The 5% methanol wash therefore appears to reduce background signal to provide better quantification. The water wash is not sufficient and it appears that formic acid either disrupts PFAS binding or the detection step.
Example 22—Investigation of a Combination Pre-Treatment of a Carbon Column with of Washing of a Hydrophobic Column Prior to Elution of Bound PFAS on the Indicator displacement Assay—A combination of the pre-treatment steps outlined in Examples 20 and 21 above was performed. The results obtained for two different environmental samples are shown in FIGS. 26A and 26B and indicate that this combination of pretreatment using both a carbon column and a hydrophobic column washed with 5% methanol did not have a significant effect for sample 1 (FIG. 26A) but did improve detection of PFAS in sample 2 (FIG. 26B). These results indicate that this combination of pre-treatment steps can be useful in some cases for improving detection of PFAS without any detrimental effects.
Example 23—Comparison of Sample Pre-Treatment with Weak Anion Exchange and Hydrophobic Solid Phase Extraction—This experiment provides a comparison of the improvement of detection of PFAS with different pre-treatment steps of WAX hydrophobic (SDVB) plate-based solid phase extraction (SPE). SPE plates were conditioned with 1 mL of methanol and equilibrated with 1 mL of MilliQ™ water. 10 mL of each sample (where samples 1 and 2 are groundwater samples and sample 3 is a leachate sample) were passed through SDVB and WAX SPE 96-well plates. The WAX plate was washed with 5% methanol and 1:10 formic acid in methanol and eluted in 1% NH₄OH in methanol. The SDVB plate was eluted with 100% methanol. Eluted samples were dried out at 90 °C and reconditioned in 100 μL of 50% methanol to achieve 100-fold concentration. Then 25 μL of sample was added to a black 96-well plate containing 25 μL of mastermix (final concentration: 0.012 mM bis-ANS, 0.015 mM pQ-β-CD and 25 mM of alpha cyclodextrin). The plate was covered and incubated at room temperature for 1 h. The fluorescence was measured using a spectrophotometer (excitation of 405 nm and emission of 510 nm). The results for the specific samples investigated in this experiment shown in FIG. 27A indicate that that the SPE hydrophobic treatment using the SDVB resin improves sensor quantification by bringing values closer to the standard curve. This may indicate that there is less retention of interferents by the column or that the interferents are not eluted in the final mix of the three samples.
Example 24—Comparison of Sample Pre-Treatment with Weak Anion Exchange and Hydrophobic Solid Phase Extraction—This experiment provides another comparison of the improvement of detection of PFAS with different pre-treatment steps of WAX hydrophobic (SDVB) plate-based solid phase extraction (SPE). Samples 1 to 3 are foamate samples and sample 4 is a leachate sample. The pre-treatments were conducted as outlined in Example 23. The results for the specific samples investigated in this experiment shown in FIG. 27B, indicate that pre-treating samples with SPE Weak Anion Exchange (WAX) resin improves sensor quantification by bringing values closer to the standard curve. This may indicate that at least some interferents are more effectively removed using WAX to provide a cleaner eluent. A comparison between the results of Example 23 and 24 provide an indication that groundwater samples may be better treated with a hydrophobic resin and that foamate samples may be better treated with WAX resin. Leachate samples may present more of a challenge for pre-treatment as a result of having more potential interferents and that detection of PFAS in such samples may benefit from both pre-treatments.
Example 25—Investigation of Mitigation of Background Fluorescence Noise Signal with Hydrophobic and WAX Pre-Treatment—In this experiment, six samples were subjected to sample pre-treatment by the hydrophobic and WAX SPE procedures outlined in Example 23. Then 25 μL of sample was added to a black 96 well plate containing 25 μL of a control mastermix to account for background noise of the system (final concentration: 0.012 mM bis-ANS, and 25 mM of alpha cyclodextrin, without the beta-cyclodextrin PQ polymer). The plate was covered and incubated at room temperature for 1 h. The fluorescence was measured using a spectrophotometer (excitation of 405 nm and emission of 510 nm). The dotted line represents the detection threshold value. The results shown in FIG. 28 indicate that there is lower non-specific fluorescence signal (background noise) in the samples treated with WAX versus SDVB. This indicate that the WAX pre-treatment will tend to provide cleaner samples and better quantification of the specific analyte and further indicates the utility of the WAX pre-treatment.
Example 26—Comparison of PFAS Detection in Samples Subjected to Pre-Treatment with Hydrophobic and WAX Pre-Treatment with PFAS Detection in Samples Analyzed using a Standard LC/MS-MS Method—A series of nine samples from different sources including leachate, foamate and groundwater were subjected to pre-treatment by hydrophobic (SDVB) and WAX SPE and subsequently analyzed using an embodiment of the internal displacement assay. The PFAS concentration determined for aliquots of each sample are shown in Table 3 below.

TABLE 3
Comparison of Detection of PFAS Samples Pre-
Treated with WAX and Hydrophobic Resins with
Detection using a Standard LC/MS-MS Method

| | | Standard | Hydrophobic | WAX |
|---|---|---|---|---|
| Sample | Matrix | (ppb) | (ppb) | (ppb) |
| 1 | Leachate | 2353 | 4.41 | 1,221 |
| 2 | Leachate | 111.79 | 92.54 | >10,000 |
| 3 | Foamate | 193.68 | 155.68 | 122.81 |
| 4 | Foamate | 235.91 | >10,000 | 299.06 |
| 5 | Foamate | 4.13 | >10,000 | 200.77 |
| 6 | Foamate | 64.18 | 11.20 | 116.05 |
| 7 | Foamate | 112.34 | 16.66 | 397.62 |
| 8 | Groundwater | 0.67 | ND | 4.56 |
| 9 | Groundwater | 0 | 1.20 | 9.07 |

The PFAS estimated concentrations closest to the expected concentration determined by the standard method are bolded and underlined. ND indicates no detection. The results indicate that foamate samples benefit from pre-treatment with WAX SPE and that groundwater samples benefit from pre-treatment with hydrophobic SPE. The results obtained for the leachate samples indicate that leachate samples may be best treated with both pre-treatment steps. It is to be understood that variability of results is to be expected in a manner depending upon the complexity of interferents present in any given sample.
Example 27—Comparison of Assay Mixtures in Dried and Liquid Form—This investigation was conducted to determine the effect of dried vs. liquid forms of a representative embodiment of an assay mixture. For the dry format, a 2 mg/mL stock solution of PFOA was prepared in 1 mL of HPLC grade methanol using glass vials. The stock solution was then used to prepare serial dilutions. After mixing and vortexing, each serial dilution was mixed 10 times with a pipette and 30 μL was pipetted out and added to a black PCR 96-well plate (Hard-Shell, Bio-Rad, Cat number: HSP9661) containing 30 μL of an indicator displacement assay mixture to provide final concentrations of 0.012 mM bis-ANS, and 0.015 mM pQ-β-CD prepared in milliQ™ water. The solution was then incubated at room temperature for 1 hour in a box to prevent photobleaching and measured using a spectrophotometer (excitation of 405 nm and emission of 510 nm). Samples were left under a fume hood for 16 hours to dry fully and then covered. The plates were then refrigerated. After 33 days, the plates were removed and 60 μL of milliQ™ water with 25% methanol (v/v) was added for reconstitution of the mixture with light mixing and left for 1 hour at room temperature. Fluorescence was then measured. For each dry plate, two rows in the plate were left empty to accommodate a comparison of the liquid format.

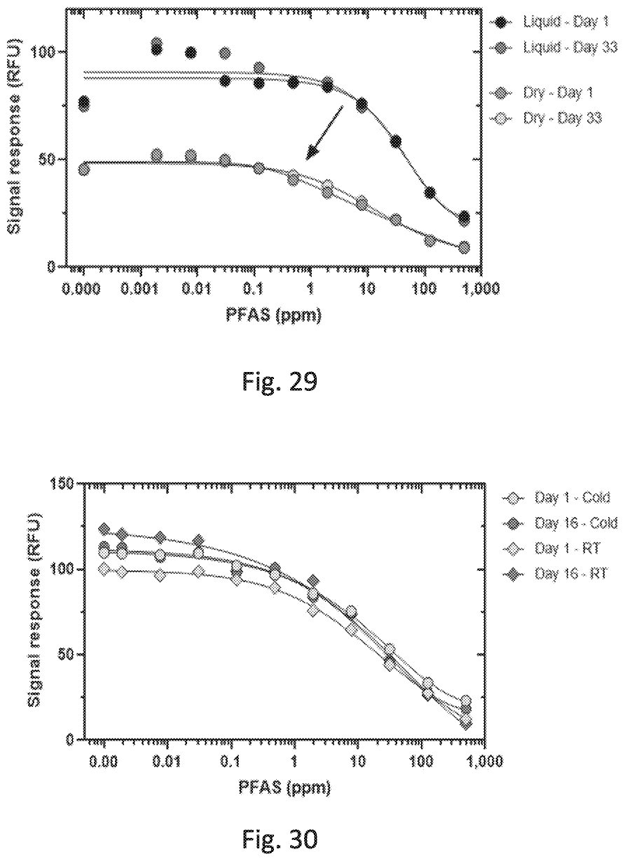

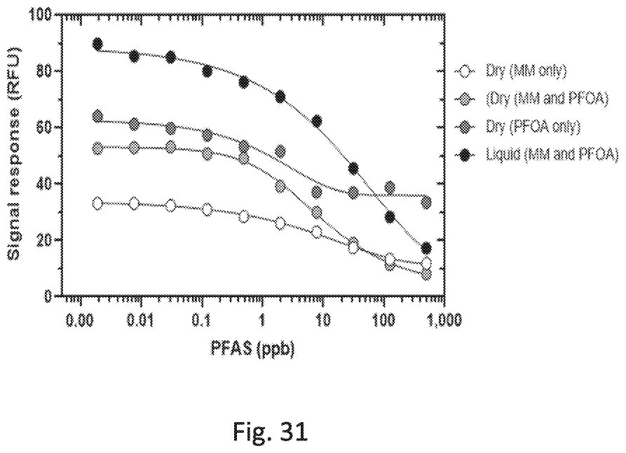

For the liquid format, the same procedure was followed except that the samples were not dried in a fume hood. FIG. 29 shows the results of the assays of the samples. While the dried format reduces the baseline fluorescence, a reliable response is observed and both the liquid and dried samples remain stable when refrigerated for 33 days.
Example 28—Comparison of Storage of an Assay Mixture with Refrigeration and at Room Temperature—The same analysis outlined in Example 27 was performed with samples either refrigerated or stored at room temperature for up to 16 days. The results shown in FIG. 30 indicate that storage of the liquid form of the assay mixture at room temperature for this period does not significantly affect the assay results.
Example 29—Comparison of drying of the Assay Mixture in the Presence and Absence of PFOA—FIG. 31 shows the results of a comparison of standard concentration curves prepared from an assay mixture dried in the presence of PFOA (pale grey circle) or absence of PFOA (white circle) and includes a comparison of the original liquid form of the mixture without drying (black circle) and in the presence of PFOA as well as dried solutions of PFOA alone (dark grey circle). For preparation of the dried versions of the assay mixture, volumes of 30 μL (final concentration: 0.012 mM bis-ANS, and 0.015 mM pQ-β-CD prepared in milliQ™ water) with or without the indicated concentrations of PFOA were dried on a Black PCR 96-wells plate (Hard-Shell, Bio-Rad, Cat number: HSP9661) by incubating the plate for 16 hours under the fume hood. For reconstitution of the dried mixtures, 60 μL of milliQ™ water with 25% methanol was added with slight mixing and incubation for 1 hour at room temperature prior to fluorescence measurements.

The results indicate that drying of the assay mixture reduces the sensitivity of the assay but still produces a viable PFAS sensor and that drying the assay mixture in the presence of the PFAS molecule PFOA provides a mixture that has improved sensitivity, as judged by the standard curves shown in FIG. 31. This surprising result indicates that production of a dried assay mixture which includes a PFAS compound improves the quality of the assay while also providing the advantage of requiring less mixing of the solutions required to prepare standard PFAS concentration curves, thereby simplifying the assay procedures. This is a particularly important advantage for field deployable assays.
Example 30—Series of Assay Solutions for Performing an Indicator Displacement PFAS Quantification Assay—In one embodiment, there is provided a kit which includes series of dried assay mixtures contained in individual wells in of a multi-well plate or in other vessels such as tubes or cuvettes for the purpose of detecting and quantifying one or more PFAS compounds in a sample. Each mixture of the series of mixtures includes the components required for the assay including a β-cyclodextrin, an indicator molecule, and optionally, a scavenger molecule such as α-cyclodextrin. Each one of the series of solutions may further include a defined concentration of a PFAS molecule, such as PFOA, for example, for the purpose of preparing a standard curve for PFAS as well as a sample analysis solution.

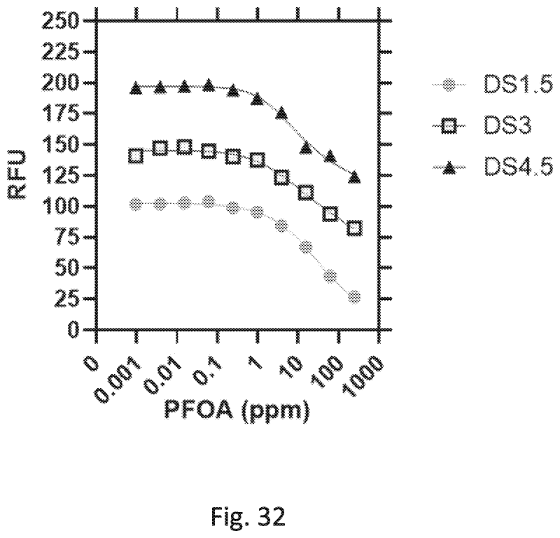

It has been established by experimentation (see Example 29) that inclusion of a PFAS molecule in an assay mixture (occasionally referred to herein as a “mastermix”) prior to drying has the effect of stabilizing the assay components for dry storage and subsequent reconstitution and that this leads to improved assay results in terms of detection sensitivity. This arrangement provides additional convenience of not requiring additional preparation of solutions while the assay is being conducted in the field, close to locations where samples are being obtained.
Example 31—Investigation of Degree of Substitution of Polymeric β-Cyclodextrin on the Indicator Displacement Assay—An investigation of the effect of increasing the substitution of the polymeric β-cyclodextrin was performed for the quaternary amine substituent, which adds positive charge to the polymeric β-cyclodextrin and which is also expected to provide an electron withdrawing effect to the glucose residues that include the substituent. Optimized mixtures of polymerized β-cyclodextrin (0.025 mM) with varying degrees of quaternary amine substitution, bis-ANS (0.012 mM), monomeric alpha cyclodextrin scavenger (10 mM), and MES buffer (pH 5) (10 mM) were combined with a dilution series of PFOA in 50% methanol (final concentration of 25% methanol). Fluorescence is plotted as a function of PFOA concentration in FIG. 32. The degree of substitution (DS) number indicates the average number of quaternary amine substituents per cyclodextrin molecule within the polymer. This experimental series compares DS1.5 (1.5 quaternary amine substitutions per cyclodextrin molecule), DS3 (3 quaternary amine substitutions per cyclodextrin molecule) and DS4.5 (4.5 quaternary amine substitutions per cyclodextrin molecule). The results indicate that the fluorescence response curves are improved when a greater degree of quaternary amine substitution is provided, thereby providing a significant advantage in increasing the sensitivity of the indicator displacement assay. This is evidenced from calculations of the approximate limit of detection of PFOA of 1 ppm for DS1.5, 0.5 ppm for DS3.0, and 0.25 ppm for DS4.5. Furthermore, the fluorescence intensity measured when the indicator molecule is bound to the polymeric β-cyclodextrin is increased with a greater degree of quaternary amine substitution. This is also a favorable characteristic for embodiments of the assay intended for use in the field. Such embodiments use portable fluorescence sensors which are typically less sensitive than expensive lab-based fluorometers. Therefore, an increase in signal is generally expected to provide an enhanced signal to noise ratio and therefore a more reliable detection of the indicator displacement by the PFAS analytes by portable fluorescence sensors.

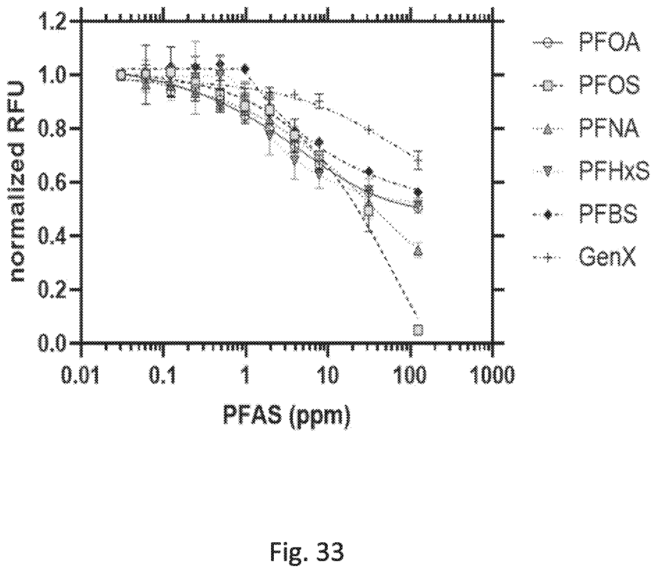

While the data presented in this example focused on quaternary amine substitution, it is reasonably predicted that other substituents providing a positive charge and/or electron withdrawing effects will provide similar results. Examples of other substituents providing such effects include, but are not limited to, a protonated primary amine substituent, a protonated secondary amine substituent, a protonated tertiary amine substituent, a cyano substituent, a nitro substituent, an aldehyde substituent, an acetyl substituent, an ester substituent, a halogen substituent, a haloalkyl substituent, a hydroxyl substituent, an alkoxyl substituent, and a sulfonyl substituent. Some embodiments of the indicator displacement assay may include polymeric β-cyclodextrin which includes a combination of any of these substituents.
Example 32—Detection of Different PFAS Molecules with Similar Limits of Detection—An investigation of the capability of the indicator displacement assay to detect six different PFAS species was undertaken using polymeric β-cyclodextrin DS4.5 (having 4.5 quaternary amine substitutions per cyclodextrin molecule of the polymer) in a reaction mixture with bis-ANS, and monomeric alpha cyclodextrin scavenger (10 mM) that was combined with dilution series of PFOA, PFOS, PFNA, PFHxS, PFBS and GenX in 50% methanol (final concentration of 25%-methanol). Fluorescence for each set was normalized to the lowest value tested and plotted as a function of concentration of each of the PFAS molecules. The results are shown in FIG. 33, where it is seen that the indicator displacement curves are generally similar for the five straight-chain PFAS molecules PFOA, PFOS, PFNA, PFHxS and PFBS. The indicator displacement curve for GenX is less responsive. It is understood that the branched structure of GenX is likely responsible for the observed difference (whereas PFOA, PFOS, PFNA, PFHxS and PFBS are straight chain PFAS molecules) and that use of the DS4.5 version of polymeric β-cyclodextrin represents an improvement with respect to consistent detection of PFOA, PFOS, PFNA, PFHxS, PFBS relative to results obtained from a similar assay using polymeric β-cyclodextrin DS1.5 (1.5 quaternary amine substitutions per cyclodextrin molecule of the polymer).

This is a useful result because it is favorable to be able to detect the most common PFAS molecules at similar limits of detection because the assay cannot distinguish between them. It is therefore helpful to be able to have similar displacement characteristics for the most common PFAS molecules, particularly when more than one PFAS compound is present and a determination of the total amount of PFAS molecules is desired. It is expected that most common applications of the indicator displacement assay will involve testing of samples obtained from sites where the identity of the PFAS molecule(s) are already known from known prior use of the PFAS molecule(s) at those sites and therefore the identities of the PFAS molecule(s) can be deduced. In other situations where the identities of the PFAS molecule(s) are not known, the assay can provide a useful estimation of “total PFAS”. The results of the total PFAS test would be similar to the results provided by the conventional EPA 1621 method which provides a measure of total adsorbable organofluorine. A separate analysis step of using conventional LC-MS/MS could then be performed to determine the identities of the PFAS molecule(s) in the sample, if desired.
Example 33—Detection of Different PFAS Molecules to Test Assay Responsiveness—An investigation of the capability of the indicator displacement assay to detect seven different PFAS compounds was undertaken using polymeric β-cyclodextrin in a reaction mixture with bis-ANS that was combined with dilution series of PFOA, PFOS, PFNA, PFHxS, PFBS, GenX and PFBA.

Stock solutions of 4 mg/mL were prepared in 1 mL of HPLC grade methanol in glass vials. The stock solutions were then used to prepare serial dilutions. After mixing and vortexing, 50 μL each serial dilution was pipetted out and added to PCR tubes containing 50 μL of an indicator displacement assay mixture containing 0.25 mM of p-β-CD combined with 0.008 mM ANS in water. The solution was then mixed using a vortex mixer and incubated at room temperature for 2 hours in a box to prevent photobleaching. Then 50 μL of each solution was transferred into a black plate. Fluorescence measurements were made using a spectrophotometer (excitation of 375 nm and emission of 477 nm). The graph shows delta fluorescence signal with control subtracted. Each experiment was repeated 3 times.

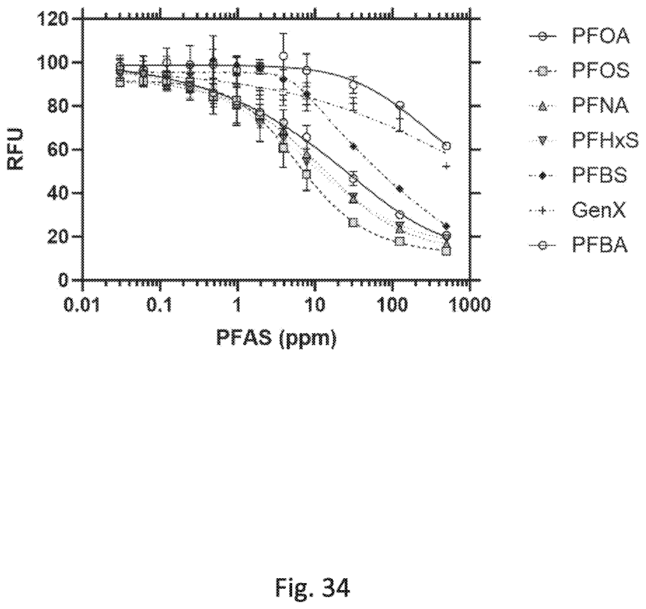

The results shown in FIG. 34 indicate that the indicator displacement curves are more responsive for PFAS compounds that have longer carbon chains, such as PFOA, PFOS, PFNA and PFBS relative to the PFAS compounds that have shorter carbon chains even as short as four carbons, such as PFBS, GenX and PFBA. While the assay is generally less responsive for the PFAS compounds with shorter carbon chains, PFBS, GenX and PFBA are still detectable and therefore embodiments of the indicator displacement assay are useful for detecting such shorter chain compounds. It is reasonably predicted that PFAS compounds as short as two carbons in length, such as trifluoroacetic acid (TFA) will also be detectable using the assay. In situations where environmental samples are being analyzed at a site where the identity of a given contaminating PFAS compound is known from historical context, knowledge of the responsiveness of the assay to individual PFAS compounds is useful information which can assist the analysis effort by providing an indication of the concentration of the contaminating PFAS compound when standard response curves are prepared for that PFAS compound and used in the analysis.
Example 34—Detection of Different PFAS Molecules with Carbon Chains Ranging from C4 to C14 to Test Assay Responsiveness—An investigation of the capability of the indicator displacement assay to detect four different PFAS compounds was undertaken using quaternary amine modified polymeric β-cyclodextrin (pQ-p-β-CD) DS1.5 in a reaction mixture with bis-ANS, and monomeric alpha cyclodextrin scavenger (10 mM) that was combined with dilution series of PFBA, PFBS, PFTDeA, and PFOA.

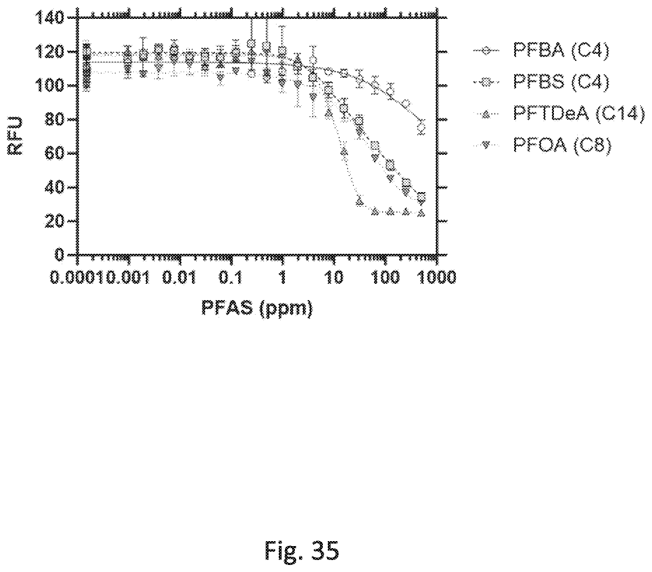

Stock solutions of 2 mg/mL stock solutions were prepared in 1 mL of HPLC grade methanol for PFOA, PFBA, PFBS and PFTDeA using glass vials. Each stock was used to prepare serial dilutions. 25 μL of each serial dilution was transferred to a black 96-well plate. Then 25 μL of the IDA mixture containing 0.012 mM BisANS (0.024 mM stock) combined with 0.015 mM pQ-β-CD (0.03 mM stock) and 20 mM m-α-CD (50 mM stock). A control was run without pQ-β-CD. The solutions were mixed by pipetting 10 times and the plate was spun down to collect droplets. The plate was then incubated at room temperature for 2 hours in a box to prevent photobleaching. After incubation, the fluorescence signal was measured using a spectrophotometer (excitation of 375 nm and emission of 477 nm). The results shown in FIG. 35 shows relative fluorescence with control subtracted. Each experiment was repeated 3 times.

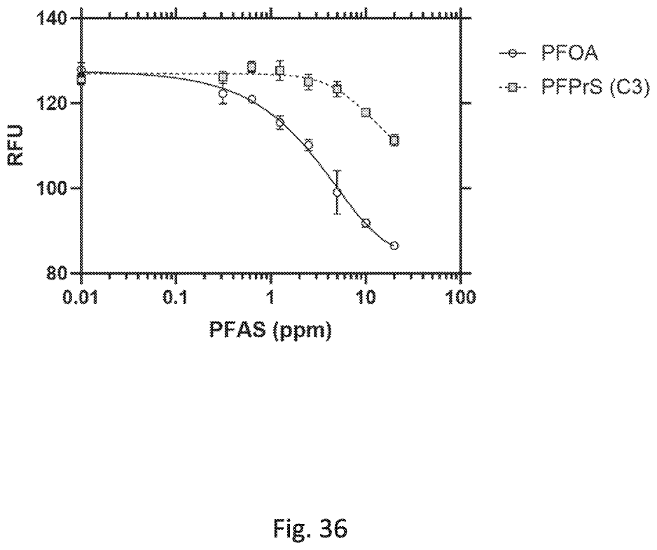

The results shown in FIG. 35 indicate that the assay responsiveness is most pronounced for PFTDeA, which has a 14-carbon chain and least pronounced for PFBA which has a 4-carbon chain. The presence of a sulfonate group in a PFAS compound appears to improve the assay responsiveness relative to a PFAS compound without a sulfonate group, as evidenced by the similar response curves for PFOA (8 carbons) and PFBS (4 carbons) as well as a comparison between the curves for PFBA and PFBS which have the same 4-carbon chain. Theory predicts that sulfonates will be the stronger binders, as they have a more stable negative functional group vs the carboxylic acids, and therefore have stronger interactions with the positively charged quaternary amines. It has been predicted that cyclodextrins will bind in a “daisy chain” arrangement to longer PFAS molecules, meaning that it is possible to have 2 or 3 cyclodextrin units bound to a PFAS molecule. This would mean 2-3 displacements per molecule, and therefore a lower limit of detection in this type of assay. This may provide a potential explanation for the more sensitive response to C14 than C4 PFAS compounds.
Example 35—Comparison of Assay Responsiveness in Detection of PFOA and PFPrS—FIG. 36 shows a comparison of the assay responsiveness for PFOA and PFPrS under similar conditions.

A PFPrS 50 ppm stock solution in 100% methanol was purchased from Wellington Laboratories Inc. Stock solutions of 2 mg/mL were prepared in 1 mL of HPLC grade methanol for PFOA using glass vials. Each stock was used to prepare dilution series. Aliquots of 25 μL of each serial dilution were transferred to a black 96-well plate. Then 25 μL of an assay mixture containing 0.012 mM BisANS (0.024 mM stock) was combined with 0.015 mM DS1.5 pQ-β-CD (0.03 mM stock) and 20 mM m-α-CD (50 mM stock). A control was run without pQ-β-CD. The solutions were mixed by pipetting 10 times and the plate was spun down to collect droplets. The plate was then incubated at room temperature for 2 hours in a box to prevent photobleaching. After incubation, the fluorescence signal was measured using a spectrophotometer (excitation of 375 nm and emission of 477 nm). The results shown in FIG. 36 shows relative fluorescence with control subtracted. Each experiment was repeated 3 times.

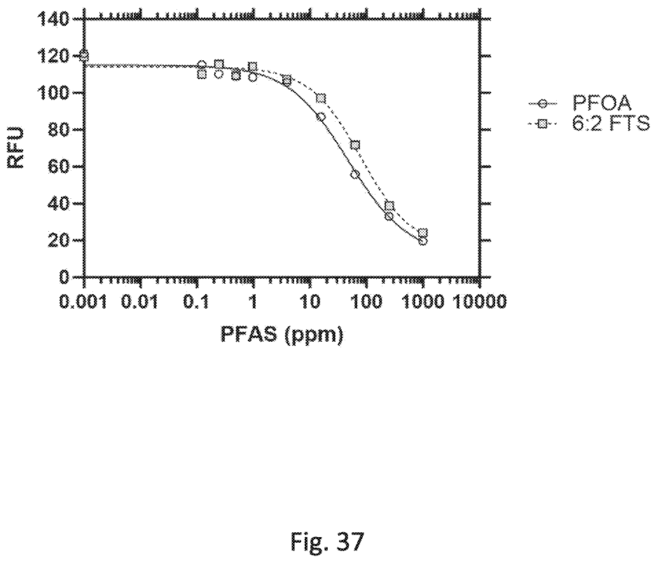

The results indicate that the assay response curve is less pronounced for PFPrS than for PFOA, as expected for a 3-carbon PFAS compound, although it contains a sulfonate group. Despite the lesser degree of responsiveness for PFPrS, the assay is expected to be useful for identifying PFPrS in environmental samples expected to contain PFPrS, particularly if it is the only PFAS compound expected to be present in the environmental sample.
Example 36—Comparison of the Limit of Detection of PFOA and 6:2 FTS—FIG. 37 shows a comparison of the assay responsiveness for PFOA and 6:2-fluorotelomersulfonic acid (6:2 FTS) under similar conditions. 6:2 FTS is a telomer, which is defined as a short polymer formed of 6 of fewer monomer units. The nomenclature “6:2” indicates that the compound has 6 fluorinated carbons linked to 2 non-fluorinated carbons.

A 250 mg/mL stock solution of 6:2 FTS was prepared in 1 mL of HPLC grade methanol using a glass vial. This stock solution was used to prepare a dilution series. After mixing and vortexing, 50 μL each serial dilution was pipetted out and added to PCR tubes containing 50 μL of the IDA mix containing 0.25 mM of p-β-CD combined with 0.008 mM ANS in water. The solution was then mixed using a vortex mixer and incubated at room temperature overnight in a box to prevent photobleaching. Each solution was then transferred into a black plate and spun down. After incubation, the fluorescence signal was measured using a spectrophotometer (excitation of 375 nm and emission of 477 nm). The results shown in FIG. 37 shows relative fluorescence with control subtracted. Each experiment was repeated 3 times.

The results indicate that the assay responsiveness is quite similar for PFOA and 6:2 FTS, as expected because both of these PFAS compounds include an 8-carbon chain.
Example 37—Detection of PFAS Compounds in Environmental Samples by an Embodiment of the Indicator Displacement Assay and a Comparison with Conventional EPA Methods—A comparison of the conventional EPA 1633 and EPA 1621 methods with an embodiment of the indicator displacement assay was performed. EPA 1621 is a modification of the total organic fluorine (TOF) measurement to make it more specific for detection of PFAS. Typical TOF measurements use combustion ion chromatography to determine the presence of organofluoride. Since such detection has a high probability of detecting fluorine originating from sources other than PFAS compounds, the total amount of fluorine detected will provide an over-estimate. The adsorbable organic fluorine (EPA method 1621-see www.epa/gov) was developed to address this issue. It involves the use of activated carbon to adsorb material and make it more selective for PFAS. The absorbed material is then subjected to combustion ion chromatography test to determine the fluorine/PFAS level.

The environmental samples investigated include semiconductor wastewater, AFFF affected groundwater and the results of rinsing equipment known to be contaminated with AFFF material (AFFF rinsate).

The values indicated in Table 4 are expressed in μg/L (ppb). The indicator displacement assay (IDA) results are the average of 3 replicates and were based on the interpolation of the IDA compared to a standard curve of known concentrations. The concentrations of individual PFAS compounds which were detected by EPA 1633 are shown in Table 5, where “<RL” indicates that the result was below the reporting limit. Of note, only the compounds that were detected via EPA 1633 are reported in Table 5 instead of the full list of 40 compounds that are detectable using this conventional assay.

TABLE 4
Comparison of Detection of PFAS Compounds

| | | | AFFF affected | | | | | |
|---|---|---|---|---|---|---|---|---|
| Semiconductor Wastewater | | | Groundwater | | | AFFF rinsate | | |
| Total PFAS ppb (μg/L) | | | Total PFAS ppb (μg/L) | | | Total PFAS ppb (μg/L) | | |
| EPA1633 | IDA | EPA1621 | EPA1633 | IDA | EPA1621 | EPA1633 | IDA | EPA1621 |
| 52.91 | 130.57 | 394 | 7048 | 6410 | 6610 | 2.59 | 2.6 | 3.8 |

TABLE 5
Detection of Individual PFAS Compounds in Environmental
Samples using Conventional EPA 1633

| | | AFFF | |
|---|---|---|---|
| | Semiconductor | Affected | AFFF |
| | Wastewater (ppt | Groundwater | Rinsate ppt |
| Analyte | (ng/L) | (ppt (ng/L) | (ng/L) |
| 6:2FTS | <RL | 2,800,000 | 2184.6 |
| 8:2FTS | <RL | <RL | 235.1 |
| NFDHA | 770 | <RL | <RL |
| PFBA | 22000 | <RL | <RL |
| PFDA | 300 | <RL | <RL |
| PFHpA | 3800 | <RL | <RL |
| PFHpS | <RL | 58,000 | <RL |
| PFHxA | 6200 | 100,000 | 170.5 |
| PFHxS | <RL | 380,000 | <RL |
| PFMBA | 1100 | <RL | <RL |
| PFMPA | 4500 | <RL | <RL |
| PFNA | 1300 | <RL | <RL |
| PFOA | 2800 | 110,000 | <RL |
| PFOS | 61 | 3,600,000 | <RL |
| PFPeA | 10000 | <RL | <RL |
| PFUnA | 77 | <RL | <RL |

In Table 5, PFAS abbreviations not previously defined are: 8:2-fluorotelomersulfonic acid (8:2 FTS); perfluoro-3,6-dioxaheptanoic acid (NFDHA); perfluorodecanoic acid (PFDA); perfluoroheptanoic acid (PFHpA); perfluoroheptanesulfonic acid (PFHpS); perfluoro-4-methyoxybutanoic acid (PFMBA); perfluoro-3-methoxypropanoic acid (PFMPA); perfluoropentanoic acid (PFPeA); and perfluoroundecanoic acid (PFUnA).

Table 4 indicates that the indicator displacement assay (IDA) provides total PFAS detection results which are generally similar to the results obtained by the two conventional EPA methods, thereby providing at least a preliminary validation of the assay for its intended purpose. Since the IDA results are generally similar to the results obtained by the conventional EPA methods, there is a basis for predicting that the indicator displacement assay will be useful for detecting any or all of the PFAS compounds listed in Table 5.

### EQUIVALENTS AND SCOPE

Other than described herein, or unless otherwise expressly specified, all numerical ranges, amounts, values and percentages, such as those for amounts of materials, elemental contents, times and current rate, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

The terms “approximately,” “about,” “substantially,” and similar terms are intended to have a broad meaning in harmony with the common and accepted usage by those of ordinary skill in the art to which the subject matter of this disclosure pertains. It should be understood by those of skill in the art that these terms are intended to allow a description of certain features described and claimed without restricting the scope of these features to the precise numeral ranges provided. Accordingly, these terms should be interpreted as indicating that insubstantial or inconsequential modifications or alterations of the subject matter described and are considered to be within the scope of the disclosure.

The articles “the”, “a” and “an” are not necessarily limited to mean only one, but rather are inclusive and open ended so as to include, optionally, multiple such elements.

“At least one,” in reference to a list of one or more entities should be understood to mean at least one entity selected from any one or more of the entities in the list of entities, but not necessarily including at least one of each and every entity specifically listed within the list of entities and not excluding any combinations of entities in the list of entities. This definition also allows that entities may optionally be present other than the entities specifically identified within the list of entities to which the phrase “at least one” refers, whether related or unrelated to those entities specifically identified. Thus, as a non-limiting example, “at least one of A and B” (or, equivalently, “at least one of A or B,” or, equivalently “at least one of A and/or B”) may refer, to at least one, optionally including more than one, A, with no B present (and optionally including entities other than B); to at least one, optionally including more than one, B, with no A present (and optionally including entities other than A); to at least one, optionally including more than one, A, and at least one, optionally including more than one, B (and optionally including other entities). In other words, the phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” may mean A alone, B alone, C alone, A and B together, A and C together, B and C together, A, B and C together, and optionally any of the above in combination with at least one other entity.

Where two or more ranges are used, such as but not limited to 1 to 5 or 2 to 4, any number between or inclusive of these ranges is implied.

As used herein, the phrase, “for example,” the phrase, “as an example,” and/or simply the term “example,” when used with reference to one or more components, features, details, structures, and/or methods according to the present disclosure, are intended to convey that the described component, feature, detail, structure, and/or method is an illustrative, non-exclusive example of components, features, details, structures, and/or methods according to the present disclosure. Thus, the described component, feature, detail, structure, and/or method is not intended to be limiting, required, or exclusive/exhaustive; and other components, features, details, structures, and/or methods, including structurally and/or functionally similar and/or equivalent components, features, details, structures, and/or methods, are also within the scope of the present disclosure.

The term “comprising” is intended to be open and permits but does not require the inclusion of additional elements or steps. When the term “comprising” is used herein, the term “consisting of” is thus also encompassed and disclosed. Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments, to the tenth of the unit of the lower limit of the range, unless the context clearly dictates otherwise. Where the term “about” is used, it is understood to reflect +/−10% of the recited value. In addition, it is to be understood that any particular embodiment that falls within the prior art may be explicitly excluded from any one or more of the claims. Since such embodiments are deemed to be known to one of ordinary skill in the art, they may be excluded even if the exclusion is not set forth explicitly herein.

In addition, it is to be understood that any particular embodiment of the present disclosure that falls within the prior art may be explicitly excluded from any one or more of the claims. Since such embodiments are deemed to be known to those of ordinary skill in the art, they may be excluded even if the exclusion is not set forth explicitly herein. Any particular embodiments of compositions disclosed herein can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

1. A method for detecting and quantifying a polyfluoroalkyl and perfluoroalkyl substance (PFAS) compound or a mixture of PFAS compounds in a sample, the method comprising:
providing a complex of a cyclodextrin and an indicator molecule; and
contacting the complex with the sample and measuring a parameter of the indicator molecule, wherein the parameter is correlated with an extent of displacement of the indicator molecule from the cyclodextrin upon binding of the PFAS compound to the cyclodextrin, thereby indicating presence and quantity of the PFAS compound or the mixture of PFAS compounds in the sample.

2. The method of claim 1, wherein the PFAS compound comprises a PFAS compound comprising at least two carbon atoms and/or the mixture of PFAS compounds comprise a mixture of PFAS compounds, each having at least two carbon atoms.

3. The method of claim 1, wherein the PFAS compound or the PFAS compound of the mixture of PFAS compounds is selected from any one of or a combination of perfluorononanoic acid (PFNA), perfluorooctanoic acid (PFOA), perfluorooctane sulfonate (PFOS), perfluorohexane sulfonate (PFHxS), perfluorohexanoic acid (PFHxA), perfluorobutane sulfonate (PFBS), perfluorobutanoic acid (PFBA), perfluorotetradecanoic acid (PFTDeA), perfluoropropanesulfonic acid (PFPrS), 6:2-fluorotelomersulfonic acid (6:2 FTS); 8:2-fluorotelomersulfonic acid (8:2 FTS); perfluoro-3,6-dioxaheptanoic acid (NFDHA); perfluorodecanoic acid (PFDA); perfluoroheptanoic acid (PFHpA); perfluoroheptanesulfonic acid (PFHpS); perfluoro-4-methyoxybutanoic acid (PFMBA); perfluoro-3-methoxypropanoic acid (PFMPA); perfluoropentanoic acid (PFPeA); trifluoroacetic acid (TFA); perfluoroundecanoic acid (PFUnA) and hexafluoropropylene oxide dimer acid (HFPO-DA), or wherein the PFAS compound is a PFAS compound of an aqueous film-forming foam (AFFF).

4. The method of claim 1, wherein the cyclodextrin is β-cyclodextrin.

5. The method of claim 1, wherein the cyclodextrin is β-cyclodextrin polymer.

6. The method of claim 5, wherein the β-cyclodextrin polymer comprises a linker.

7. The method of claim 6, wherein the linker is a cyclic ether.

8. The method of claim 7, wherein the cyclic ether is a substituted or unsubstituted oxacyclopropane, oxacyclobutane, oxacyclopentane, oxacyclohexane or furan.

9. The method of claim 5, wherein the β-cyclodextrin polymer comprises at least one non-hydrogen substituent on at least one glucopyranoside unit.

10. The method of claim 9, wherein the non-hydrogen substituent is an electron withdrawing group and/or a positively charged substituent.

11. The method of claim 9, wherein the non-hydrogen substituent is selected from any one of or a combination of: protonated primary amine, protonated secondary amine, protonated tertiary amine, quaternary amine, cyano, azido, nitro, hydroxypropyl, hydroxyamino, aldehyde, carboxyl, acetyl, ester, halogen, haloalkyl, hydroxyl, alkoxyl, and sulfonyl.

12. The method of claim 9, wherein the β-cyclodextrin polymer comprises an average of at least about 1.5 quaternary amine substituents per cyclodextrin unit, at least about 3 quaternary amine substituents per cyclodextrin unit, or at least about 4.5 quaternary amine substituents per cyclodextrin unit.

13. The method of claim 1, wherein the indicator molecule is a dye molecule.

14. The method of claim 13, wherein the dye molecule is a colorimetric dye molecule or a fluorescent dye molecule.

15. The method of claim 14, wherein the fluorescent dye molecule is 9-(diethylamino)-5H-benzo[a]phenoxazin-5-one) (Nile red), SYPRO™ Orange, 8-anilinonaphthalene-1-sulfonic acid (ANS) or bis-8-anilinonaphthalene-1-sulfonic acid (bis-ANS).

16. The method of claim 1, wherein the sample is processed by column chromatography, and resuspension in a reduced volume of solvent to concentrate the sample prior to the step of contacting the complex with the sample.

17. The method of claim 1, further comprising providing a scavenger molecule for sequestering one or more interferents present in the sample.

18. The method of claim 17, wherein the interferents are compounds having at a hydrocarbon chain of at least 6 carbons.

19. The method of claim 17, wherein the interferents are hydrocarbons or surfactants.

20. The method of claim 17, wherein the scavenger molecule is α-cyclodextrin.
